(19) United States
(12) Patent Application Publication
(10) Pub. No.: US 2024/0392197 A1
(43) Pub. Date: Nov. 28, 2024

(54) Pyrolysis Processes for Upgrading a Hydrocarbon Feed

(71) Applicant: ExxonMobil Chemical Patents Inc., Baytown, TX (US)

(72) Inventors: Mark A. Nierode, Kingwood, TX (US); Rodney S. Smith, Edinburgh (GB); Michael A. Radzicki, Houston, TX (US); Donald J. Norris, London, Ontario (CA); Kapil Kandel, Humble, TX (US); Tania M. Almazan, Baton Rouge, LA (US)

(21) Appl. No.: 18/691,105

(22) PCT Filed: Oct. 3, 2022
(86) PCT No.: PCT/US2022/077461

§ 371 Date: Mar. 12, 2024

Related U.S. Application Data
(60) Provisional application No. 63/253,167, filed on Oct. 7, 2021.

(51) Int. Cl. C10G 9/36; B01D 53/047; B01D 53/14; C01B 3/56; C10G 55/04; C10K 3/04

(52) U.S. Cl. CPC C10G 9/36; B01D 53/047; B01D 53/1456; C01B 3/56; C10G 55/04; C10K 3/04; C01B 2203/0445

(57) ABSTRACT

Processes for upgrading a hydrocarbon for a predetermined period of time. The process can include determining an amount of one or more contaminant-containing compositions that will be present in a pyrolysis effluent based, at least in part, on a composition of a hydrocarbon feed to be steam cracked, a. temperature the hydrocarbon feed will be heated at during steam cracking, a residence time the hydrocarbon feed will be heated at the temperature during steam cracking, or a combination thereof. In some examples, the process can also include taking one or more steps to allow the hydrocarbon feed to be steam cracked for at least as long as a predetermined, period of time, such as controlling process conditions within one or more separation stages to favor certain product compositions and/or introducing a predetermined amount of one or more materials into various locations of the process, or any combination thereof.

### CROSS-REFERENCE TO RELATED APPLICATION

This application claims the priority to and benefit of U.S. Provisional Patent Application 63/253,167 filed 7 Oct. 2021 entitled “PYROLYSIS PROCESSES FOR UPGRADING A HYDROCARBON FEED,” the content of which is incorporated by reference herein in its entirety.

### FIELD

Embodiments disclosed herein generally relate to pyrolysis processes for upgrading a hydrocarbon feed. More particularly, such processes relate to pyrolysis processes for pyrolysing a hydrocarbon feed that produces a pyrolysis effluent containing one or more contaminant-containing compositions.

### BACKGROUND

Pyrolysis processes, e.g., steam cracking, convert saturated hydrocarbons, e.g., paraffins, to higher-value products, e.g., light olefins such as ethylene and propylene. In addition to these higher-value products, however, the pyrolysis process also produces naphtha, gas oil, and a significant amount of relatively low-value heavy products such as pyrolysis tar. In a steam cracking process, a primary separator is typically used to separate the various products, such as a process gas, a steam cracker naphtha (SCN) or “pygas”, a steam cracker gas oil (SCGO), a steam cracker quench oil (SCQO), a steam cracker tar (SCT), etc, from a steam cracker effluent.

It has become increasingly desirable to utilize low value raw feedstocks, e.g., a C₅₊ hydrocarbon, as the hydrocarbon feed to the pyrolysis unit, e.g., steam cracker. While these raw feedstocks are attractive from a cost standpoint, such feedstocks can introduce significant levels of contaminant-containing compositions, e.g., oxygenates, into the pyrolysis process that typically have not been a concern in conventional pyrolysis processes that utilize higher value feedstocks, e.g., C₂-C₄ hydrocarbons.

There is a need, therefore, for improved pyrolysis processes, e.g., steam cracking, for upgrading a hydrocarbon feed that includes one or more contaminant-containing compositions and/or produces one or more contaminant-containing compositions during pyrolysis thereof.

### SUMMARY

Processes for upgrading a hydrocarbon are provided. In some examples, the process for upgrading a hydrocarbon can include determining an amount of one or more oxygen-containing contaminants, such as acetic acid, that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof. The hydrocarbon feed can be steam cracked to produce the steam cracker effluent. A tar product, a steam cracker quench oil, and a first overhead can be separated from the steam cracker effluent, wherein the first overhead comprises pygas, acetic acid, and a process gas comprising ethylene. A second overhead that can include the process gas and a first naphtha cut that can include the pygas, water, and acetic acid can be separated from the first overhead. A pygas product and a first aqueous mixture that can include acetic acid can be separated from the first naphtha cut. The first aqueous mixture can be contacted with a predetermined amount of a neutralizing agent sufficient to produce a treated mixture comprising neutralized acetic acid.

In some examples, the process for upgrading a hydrocarbon, can include determining an amount of carbon monoxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof. The hydrocarbon feed can be steam cracked to produce the steam cracker effluent. A tar product, a steam cracker quench oil, and a first overhead can be separated from the steam cracker effluent. The first overhead can include pygas and a process gas that can include ethylene and carbon monoxide. A second overhead that can include the process gas and a naphtha cut that can include the pygas can be separated from the first overhead. A hydrogen-rich gas can be separated from the second overhead. The hydrogen-rich gas can include a first portion of the carbon monoxide contained in the steam cracker effluent. In some examples, the hydrogen-rich gas can be introduced into a methanator having a predetermined size that is sufficient to convert a majority, preferably substantially all of the first portion of carbon monoxide to methane. In other examples, the hydrogen-rich gas can be introduced into a pressure swing adsorption unit having a predetermined size that is sufficient to remove a majority, preferably substantially all of the first portion of carbon monoxide from the hydrogen-rich gas. In still other examples, a first portion of the hydrogen-rich gas can be introduced into the methanator and a second portion of the hydrogen-rich gas can be introduced into the pressure swing adsorption unit.

The hydrocarbon feed can be steam cracked to produce the steam cracker effluent. A tar product, a steam cracker quench oil, and a first overhead can be separated from the steam cracker effluent. The first overhead can include pygas and a process gas. A second overhead that can include the process gas and carbon dioxide and a naphtha cut that can include the pygas can be separated from the first overhead. The second overhead can be contacted with an amine within the amine unit having the predetermined size to remove at least 75 wt % of the carbon dioxide in the second overhead by producing a spent amine comprising a reaction product of the amine and the carbon dioxide. A third overhead that can include ethylene and carbon dioxide and a second bottoms that can include the spent amine can be separated from the amine unit. The second bottoms can be introduced into the amine regeneration unit to produce a regenerated amine and carbon dioxide.

In some examples, the process for upgrading a hydrocarbon can include determining an amount of carbon dioxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof. An amine unit having a predetermined capacity and/or size can be installed according to the predetermined amount of carbon dioxide and other acidic gases (e.g., H₂S) in the steam cracker effluent. The amine unit can be configured to remove a minimum amount of carbon dioxide from a second overhead that includes ethylene and carbon dioxide. The predetermined capacity of the amine unit can be based, at least in part, on the determined amount of carbon dioxide that will be present in the steam cracker effluent. An amine regeneration unit having a predetermined capacity and/or size can be installed. The amine regeneration unit can be configured to regenerate a spent amine produced in the amine unit.

In some examples, a process for upgrading a hydrocarbon can include determining an amount of carbon dioxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof. An aqueous amine solution having a predetermined concentration of amine can be prepared. The predetermined concentration of the amine in the aqueous amine solution can be based, at least in part, on the determined amount of carbon dioxide that will be present in the steam cracker effluent and the predetermined size of the amine unit. The concentration of the amine in the aqueous amine solution can be adjusted and optimized where the feed and/or the steam cracking conditions change. The hydrocarbon feed can be steam cracked to produce a steam cracker effluent. A tar product, a steam cracker quench oil, and a first overhead can be separated from the steam cracker effluent. The first overhead can include pygas and a process gas that can include ethylene and carbon dioxide. A second overhead that can include the process gas and carbon dioxide and a first naphtha cut that can include the pygas can be separated from the first 5 overhead. The second overhead can be contacted with the aqueous amine solution having the predetermined concentration of amine to produce a treated mixture that can include an amine treated second overhead and a spent aqueous amine comprising an amine salt. A third overhead that can include the amine treated second overhead and a second bottoms cut that can include the spent aqueous amine can be separated from the treated mixture. At least 75 wt % of the carbon dioxide in the process gas can be removed with the spent aqueous amine in the form of the amine salt.

In some examples, a process for upgrading a hydrocarbon can include determining an amount of carbon dioxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof. A sufficient amount of a sorbent can be introduced into a carbonyl sulfide removal unit to allow the carbonyl sulfide removal unit to process a depropanizer overhead that can be separated from the steam cracker effluent for at least as long as a predetermined period of time without requiring replacement or re-activation of the sorbent due to deactivation caused by carbon dioxide present in the depropanizer overhead.

In some examples, a process for upgrading a hydrocarbon can include determining an amount of carbon dioxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts a schematic of an illustrative system for steam cracking a hydrocarbon feed to produce a steam cracker effluent and accounting for or otherwise handling acetic acid contained therein, according to one or more embodiments described.

FIG. 2 depicts a schematic of an illustrative system for steam cracking a hydrocarbon feed to produce a steam cracker effluent and accounting for or otherwise handling carbon monoxide contained therein, according to one or more embodiments described.

FIG. 3 depicts a schematic of an illustrative system for steam cracking a hydrocarbon feed to produce a steam cracker effluent and accounting for or otherwise handling carbon dioxide contained therein, according to one or more embodiments described.

FIG. 4 depicts a schematic of an illustrative system for steam cracking a hydrocarbon feed to produce a steam cracker effluent and accounting for or otherwise handling carbon dioxide contained therein, according to one or more embodiments described.

### DETAILED DESCRIPTION

It is to be understood that the following disclosure describes several exemplary embodiments for implementing different features, structures, and/or functions of the invention. Exemplary embodiments of components, arrangements, and configurations are described below to simplify the present disclosure; however, these exemplary embodiments are provided merely as examples and are not intended to limit the scope of the invention. Additionally, the present disclosure may repeat reference numerals and/or letters in the various exemplary embodiments and across the Figures provided herein. This repetition is for the purpose of simplicity and clarity and does not in itself dictate a relationship between the various exemplary embodiments and/or configurations discussed in the Figures. Moreover, the exemplary embodiments presented below can be combined in any combination of ways, i.e., any element from one exemplary embodiment can be used in any other exemplary embodiment, without departing from the scope of the disclosure.

It has been discovered that a pyrolysis effluent produced by pyrolysing a hydrocarbon feed, e.g., crude oil or a fraction thereof, can include a number of contaminant-containing compositions that can cause process disruptions, e.g., saturation of a sorbent and/or fouling, and even lead to a shutdown of the pyrolysis system. For simplicity and ease of description the pyrolysis system and effluent will be further discussed and described herein in the context of a steam cracker process and plant that produces a steam cracker effluent that includes cracked hydrocarbons and one or more contaminant-containing compositions.

The contaminant-containing composition can be or can include one or more compounds that include oxygen. The contaminant-containing composition can be or can include, but is not limited to, acetic acid, carbon monoxide, carbon dioxide, or any mixture thereof. It has also been discovered that the amount of the contaminant-containing composition that will be present in the steam cracker effluent can be determined based, at least in part, on a composition of a hydrocarbon feed to be steam cracked, a temperature the hydrocarbon feed will be heated at during steam cracking, a residence time the hydrocarbon feed will be heated at the temperature during steam cracking, or any combination thereof.

“Hydrocarbon” means a class of compounds containing hydrogen bound to carbon. The term “Cₙ” hydrocarbon means hydrocarbon having n carbon atom(s) per molecule, where n is a positive integer. The term “Cₙ₊” hydrocarbon means hydrocarbon having at least n carbon atom(s) per molecule, where n is a positive integer. The term “Cₙ₋” hydrocarbon means hydrocarbon having no more than n number of carbon atom(s) per molecule, where n is a positive integer. “Hydrocarbon” encompasses (i) saturated hydrocarbon, (ii) unsaturated hydrocarbon, and (iii) mixtures of hydrocarbons, including mixtures of hydrocarbon compounds (saturated and/or unsaturated), including mixtures of hydrocarbon compounds having different values of n. “Hydrocarbon feed” means an input into a pyrolysis process that includes hydrocarbon.

“Naphthenic acid” means a C7-C20 carboxylic acid comprising at least one carboxyl group (—COOH), including but not limited to those having a cyclopentyl and/or a cyclohexyl ring in its molecular structure. An exemplary naphthenic acid is

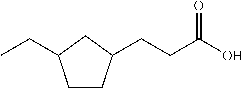

(3-(3-ethylcyclopentyl)propanoic acid).

As used herein. “neutralized acetic acid” means a salt or a compound of neutralized acid. Examples of neutralized acetic acid include, but not limited to: sodium acetate, potassium acetate, ammonium acetate, and the like.

Nomenclature of elements and groups thereof refer to the Periodic Table used by the International Union of Pure and Applied Chemistry after 1988. An example of the Periodic Table is shown in the inner page of the front cover of Advanced Inorganic Chemistry, 6^{th} Edition, by F. Albert Cotton et al. (John Wiley & Sons, Inc., 1999).

Typically, the hydrocarbon feed does not contain an appreciable amount of acetic acid, carbon monoxide, and carbon dioxide. Those skilled in the art will appreciate that a major amount, and typically substantially all, of these three compounds as may be present in the steam cracker effluent are produced in the steam cracking furnace. Acetic acid, carbon monoxide, and carbon dioxide are primarily produced during steam cracking of the hydrocarbon feed and/or decoking of the steam cracker furnace. One source for the acetic acid, carbon monoxide, and carbon dioxide can be derived from during steam cracking of naphthenic acids and other compounds in the hydrocarbon feed. The conversion or decomposition of naphthenic acids can be high in the stream cracker, e.g., ≥95%, to light acids (primarily acetic acid), carbon monoxide, and carbon dioxide. The hydrocarbon feeds that include naphthenic acids are often described by the total acid number “TAN” as an indicator of the amount of naphthenic acid(s) therein, and can have a TAN of ≥0.5 mg KOH/gram of hydrocarbon feed, ≥1 mg KOH/gram of hydrocarbon feed, ≥1.5 mg KOH/gram of hydrocarbon feed, or ≥2 mg KOH/gram of hydrocarbon feed, which can be measured for a given hydrocarbon feed according to by ASTM D664-18e2. Carbon monoxide can also be produced by methanol decomposing in the steam cracker and during decoking operations of the steam cracker. It has also been observed that CO is produced on freshly de-coked furnace tubes for a period of time (e.g. 1-3 hours) after the steam cracking furnace is brought back into pyrolysis mode. The processes of this disclosure are particularly advantageous in steam cracking of hydrocarbon feeds having a TAN≥0.5 mg KOH/gram of the hydrocarbon feed. For the sake of brevity, in this disclosure, unless indicated otherwise, the unit of all TAN data is mg KOH per gram of the hydrocarbon feed.

It has been discovered that steam cracking a hydrocarbon feed which can be a crude oil or a fraction thereof can produce a stream cracker effluent comprising acetic acid, CO, and CO₂ at appreciable concentrations, e.g. based on the total weight of the steam cracker effluent; (i) from 50, 60, 70, 80, 90 ppm by weight, to 100, 200, 300, 400, 500 ppm by weight, to 600, 700, 800, 900, 1,000 ppm by weight, to 1,500, 2,000, 2,500, 3,000, 3,500 ppm by weight, to 4.000, 4.500, or 5.000 ppm by weight, of acetic acid; (ii) from 50, 60, 70, 80, 90 ppm by weight, to 100, 200, 300, 400, 500 ppm by weight to 600, 700, 800, 900, 1,000 ppm by weight, to 1,500, 2,000, 2,500, 3,000, 3,500 ppm by weight, to 4,000, 4,500, or 5,000 ppm by weight, of CO₂ and (iii) from 50, 60, 70, 80, 90 ppm by weight, to 100, 200, 300, 400, 500 ppm by weight, to 600, 700, 800, 900, 1,000 ppm by weight, to 1,500, 2,000, 2,500, 3,000, 3,500 ppm by weight, to 4,000, 4,500, or 5,000 ppm by weight, of CO₂.

The composition of the hydrocarbon feed can be determined using one or more of a number of standardized tests that measure a compositional property or other property of the hydrocarbon feed. In some examples, the composition of the hydrocarbon feed can be determined by determining a total acid number (TAN), e.g., ASTM D664-18e2; a heptane insoluble asphaltene content, e.g., ASTM D6560-17; a carbon, hydrogen, and nitrogen content, e.g., ASTM D5291-16; a carbon residue, e.g., ASTM D4530-15 and/or ASTM D524-15; a density, a relative density, and/or API gravity, e.g., ASTM D4052-18a; distillation fractions, e.g., ASTM D2892-18a and/or ASTM D5236-18a; speciation and quantification of light hydrocarbons and cut point intervals in the hydrocarbon feed, e.g., ASTM D8003-15a; an amount of dissolved hydrogen sulfide, e.g., ASTM D7621-16; an amount of basic organic nitrogen, e.g., UOP269-10; an amount of phosphorus, e.g., ASTM D7111-16, ASTM D5185-18, ASTM D7691-16; a pour point, e.g., ASTM D5853-17a; an amount of salt, e.g., ASTM D3230-19 and/or ASTM D6470-99 (2015); total sediment particulates, e.g. ASTM D4007-11 (2016)e1 and/or ASTM D4807-05 (2015); an amount of mercaptan sulfur, e.g., ASTM D3227-16 and/or UOP163-10; a total amount of sulfur, e.g., ASTM D4294-16e1; an amount of trace metals, e.g., ASTM D5708-15, ASTM D5863-00a (2016), and/or ASTM D7691-16; a UOP K factor, e.g., UOP375-07; a vapor pressure, e.g., ASTM D323-15a and/or ASTM D6377-16, a viscosity, e.g., ASTM D445-18, ASTM D446-12 (2017), and/or ASTM D7042-16e3; an amount of water, e.g., ASTM D4377-00 (2011) and/or ASTM D4928-12 (2018); an amount of wax, e.g., UOP46-85; or any combination thereof.

In some embodiments, the hydrocarbon feed subjected to stream cracking in the processes of this disclosure can be a raw feed (e.g., a desalted crude oil without substantial separation), comprising naphthenic acid(s) at various concentrations. In other, preferred embodiments, the hydrocarbon feed subjected to stream cracking can be a fraction of a raw feed, particularly where the raw feed has a high concentration of total naphthenic acid(s) indicated by a high TAN≥0.5, or ≥1.0, or ≥1.5, or ≥2.0, or ≥2.5, or ≥3.0 mg KOH per gram of the hydrocarbon feed. In those particularly preferred embodiments, a raw feed having a TAN ≥0.5 may be separated in a flashing drum to produce an overhead vapor and a heavy bottoms liquid, and the overhead vapor or a portion thereof is suppled as at least a portion of the hydrocarbon feed subjected to steam cracking. In these embodiments, a significant portion, ≥ 5 wt % (e.g., ≥8 wt %, ≥10 wt %, ≥12 wt %, ≥14 wt %, ≥15 wt %, ≥16 wt %, ≥18 wt %, and up to 20 wt %) of the naphthenic acid(s) contained in the raw feed can be distributed into the heavy bottoms liquid. Therefore, only a portion of the naphthenic acids contained in the raw feed is distributed into the overhead vapor and then supplied into the steam cracker, resulting in the production of a smaller quantity of acetic acid in the steam cracker effluent and a reduced need to separate/process acetic acid in the separation/recovery process, compared to feeding the total composition of the raw feed into the steam cracker without separation in the flashing drum. Flashing drums used for separating a raw feed is sometimes called a k-pot or a Kuhn Pot. Exemplary flashing drums and flashing processes useful for separating such raw feed can be found in, e.g., U.S. Pat. Nos. 7,138,047; 7,767,008; 8,158,840; and 8,277,639, the relevant contents thereof are hereby incorporated by reference.

In some examples, depending on the particular contaminant-containing composition at issue, one or more steps can be taken to allow the steam cracking process to run for a predetermined period of time before requiring shutdown due to a particular contaminant-containing composition contained in the hydrocarbon feed and/or produced during steam cracking of the hydrocarbon feed. In some examples, the predetermined step(s) can include, but are not limited to, contacting certain process streams with a predetermined amount of one or more materials, e.g., neutralizing agents, aqueous amine solutions, and/or caustic solutions, where the neutralizing agent, amine, and caustic are present in a sufficient amount to neutralize or otherwise interact with and render the particular contaminant-containing composition removable from a given process stream. For example, a sufficient amount of a material can react with a particular contaminant-containing composition to produce a salt that can be separated from a particular process steam. In other examples, the predetermined step(s) can include introducing a sufficient amount of a sorbent, a catalyst, or other component into one or more process stages, e.g., separation stages or reactor stages, separating a certain contaminant-containing composition from the process at one or more predetermined locations, installing one or more separation stages having a predetermined size configured to separate out a predetermined amount of a contaminant-containing composition or a product derived therefrom from a particular process steam, and/or installing one or more reactor stages having a predetermined size configured to convert a predetermined amount of a contaminant-containing composition or a product derived therefrom in particular process steam to a different compound.

The predetermined period of time the steam cracking process can be configured to run for can be any desired length of time. In some examples, the predetermined period of time can be about 1 day, about 2 days, or about 3 days to about 1 month, about 6 months, about 1 year, about 1.5 years, about 2 years, about 3 years, or about 4 years. In some examples, the predetermined period of time can be based, at least in part, on a desired volume of the hydrocarbon feed that is to be steam cracked during the predetermined period of time.

The hydrocarbon feed, e.g., a C₅₊ hydrocarbon, can be mixed, blended, combined, or otherwise contacted with water, steam, or a mixture thereof and heated, e.g., to a temperature of about 200° C., to about 585° C., to produce a heated mixture. For example, the hydrocarbon feed can be heated by indirect heat exchange within a convection section of a steam cracker.

Hydrocarbon feeds that can be mixed, blended, combined, or otherwise contacted with the water and/or steam and heated to produce the heated mixture can be or can include, but are not limited to, raw crude oil, desalted crude oil, gas oils, heating oil, jet fuel, diesel, kerosene, gasoline, coker naphtha, steam cracked naphtha, catalytically cracked naphtha, hydrocrackate, reformate, raffinate reformate, Fischer-Tropsch liquids, Fischer-Tropsch gases, natural gasoline, distillate, virgin naphtha, atmospheric pipestill bottoms, vacuum pipestill streams such as vacuum pipestill bottoms and wide boiling range vacuum pipestill naphtha to gas oil condensates, heavy non-virgin hydrocarbons from refineries, vacuum gas oils, heavy gas oil, naphtha contaminated with crude, atmospheric residue, heavy residue, a C4/residue admixture, naphtha/residue admixture, hydrocarbon gases/residue admixture, hydrogen/residue admixtures, waxy residues, gas oil/residue admixture, fractions thereof, or any mixture thereof. In other examples, the hydrocarbon feed can be or include, naphtha, gas oil, vacuum gas oil, waxy residues, atmospheric residues, residue admixtures, crude oil, or any mixture thereof. In some examples, a crude oil fraction can be produced by separating atmospheric pipestill “APS” bottoms from a crude oil followed by vacuum pipestill “VPS” treatment of the APS bottoms. In some examples, the hydrocarbon feed can be or include a crude oil such as a high-sulfur virgin crude oil rich in polycyclic aromatics or a fraction thereof. In other examples, the hydrocarbon feed can be or include a hydroprocessed hydrocarbon, e.g., a crude or resid-containing fraction thereof. In other examples, the hydrocarbon feed can be or include a vapor phase separated from a vacuum resid subjected to a thermal conversion process in a thermal conversion reactor, e.g., a delayed coker, a fluid coker, a flex-coker, a visbreaker, and/or a catalytic hydrovisbreaker). In still other examples, the hydrocarbon feed can be can include hydrocarbons having a high TAN, e.g., a TAN of ≥0.5, ≥1, ≥1.5, or ≥2, as determined according to ASTM D664-18e2. In at least some examples, the hydrocarbon feed can be or can include, but is not limited to, naphtha, gas oil, vacuum gas oil, a waxy residue, an atmospheric residue, a crude oil, a fraction thereof, or a mixture thereof. In some examples, if a raw crude oil or other hydrocarbon that includes salts will be steam cracked, the raw crude oil or other hydrocarbon can optionally be subjected to pretreatment, e.g., desalting, to remove at least a portion of any salts contained in the raw crude oil or other hydrocarbon before heating the hydrocarbon feed to produce the heated mixture.

“Raw” feedstock or feed, e.g., raw hydrocarbon feedstock, means a primarily liquid-phase feedstock that includes ≥25 wt % of crude oil that has not been subjected to prior desalting and/or prior fractionation with reflux, e.g., ≥50 wt %, such as ≥75 wt %, or ≥90 wt %. “Crude oil” means a mixture comprising naturally-occurring hydrocarbon of geological origin, where the mixture (i) includes ≥1 wt % of resid, e.g. ≥5 wt %, such as ≥10 wt %, and (ii) has an API gravity ≤52°, e.g., ≤30°, such as ≤20°, or ≤10°, or <8°. The crude oil can be classified by API gravity, e.g., heavy crude oil has an API gravity in the range of from 5° up to (but not including) 22°.

Certain medium and/or heavy hydrocarbons, e.g., certain raw hydrocarbon feedstocks, such as certain crude oils and crude oil mixtures contain one or more of asphaltenes, precursors of asphaltenes, and particulates. Asphaltenes are described in U.S. Pat. No. 5,871,634. Asphaltene content can be determined using ASTM D6560-17. Asphaltenes in the hydrocarbon can be in the liquid phase (e.g., a miscible liquid phase), and also in a solid and/or semi-solid phase (e.g., as a precipitate). Asphaltenes and asphaltene precursors are typically present in a crude oil's resid portion. “Resid” means an oleaginous mixture, typically contained in or derived from crude oil, the mixture having a normal boiling point range ≥566° C. Resid can include “non-volatile components”, meaning compositions (organic and/or inorganic) having a normal boiling point range ≥590° C. Non-volatile components may be further limited to components with a boiling point of about 760° C., or greater. Non-volatile components can include coke precursors, which are moderately heavy and/or reactive molecules, such as multi-ring aromatic compounds, which can condense from the vapor phase and then form coke under the specified steam cracking conditions. Medium and/or heavy hydrocarbons (particularly the resid portion thereof) can also contain particulates, meaning solids and/or semi-solids in particle form. Particulates may be organic and/or inorganic, and can include coke, ash, sand, precipitated salts, etc. Although precipitated asphaltenes may be solid or semi-solid, precipitated asphaltenes are considered to be in the class of asphaltenes, not in the class of particulates.

In some examples, the hydrocarbon feed that can be mixed, blended, combined, or otherwise contacted with the water and/or steam and heated to produce the heated mixture can be or include the hydrocarbons or hydrocarbon feeds disclosed in U.S. Pat. Nos. 7,993,435; 8,277,639; 8,696,888; 9,327,260; 9,637,694; 9,657,239; and 9,777,227; and International Patent Application Publication No. WO 2018/111574.

The heated mixture can be subjected to steam cracking conditions to produce a steam cracker effluent. In some examples, a vapor phase product or first vapor phase product and a liquid phase product or first liquid phase product can be separated from the heated mixture before subjecting the heated mixture to steam cracking by introducing the heated mixture into one or more hydrocarbon feed separation stages. The vapor phase product can be heated to a temperature of ≥400° C., e.g., a temperature of about 425° C., to about 825° C., and subjected to steam cracking conditions to produce the steam cracker effluent. In some examples, the optional hydrocarbon feed separation stage can be or include the separators and/or other equipment disclosed in U.S. Pat. Nos. 7,138,047; 7,090,765; 7,097,758; 7,820,035; 7,311,746; 7,220,887; 7,244,871; 7,247,765; 7,351,872; 7,297,833; 7,488,459; 7,312,371; 6,632,351; 7,578,929; 7,235,705; and 8,158,840.

The steam cracking conditions can include, but are not limited to, one or more of: exposing the hydrocarbon feed to a temperature (as measured at a radiant outlet of a steam cracking apparatus) of ≥400° C., e.g., a temperature of about 700° C., about 800° C., or about 900° C., to about 950° C., about 1,000° C., or about 1050° C., a pressure of about 0.1 bar to about 5 bars (absolute), and/or a steam cracking residence time of about 0.01 seconds to about 5 seconds. In some examples, the hydrocarbon feed can be steam cracked according to the processes and systems disclosed in U.S. Pat. Nos. 6,419,885; 7,993,435; 9,637,694; and 9,777,227; U.S. Patent Application Publication No. 2018/0170832; and International Patent Application Publication No. WO 2018/111574.

The steam cracker effluent can be at a temperature of ≥300° C. ≥400° C., ≥500° C., ≥600° C., or ≥700° C., or ≥800° C., or more. The steam cracker effluent can be cooled to produce a cooled steam cracker effluent. For example, the steam cracker effluent can be directly contacted with an optional quench fluid and/or indirectly cooled via one or more heat exchangers, e.g., a transfer line exchanger “TLE”, to produce the cooled steam cracker effluent.

Those skilled in the art will appreciate that the amount of the optional quench fluid contacted with the steam cracker effluent should be sufficient to cool the steam cracker effluent to facilitate separation of desired products therefrom. In some examples, the steam cracker effluent can be cooled to a temperature of ≤300° C., e.g., about 160° C., to about 250° C., which can minimize or reduce fouling within one or more separation or other process equipment due to reactive compounds in the steam cracker effluent. Although the amount of quench fluid needed to do this can vary considerably from facility to facility, the quench fluid to steam cracker effluent weight ratio is typically in the range of from about 0.1 to about 10, e.g., 0.5 to 5, such as 1 to 4. The desired weight ratio in a particular instance can be determined, e.g., from any one or more of a number of factors such as the amount of steam cracker effluent to be cooled, the temperature of the steam cracker effluent at the quenching location, the composition and thermodynamic properties (e.g., enthalpy, C_{P}, etc.) of the quench fluid and the steam cracker effluent, the desired temperature of the quench fluid-steam cracker effluent mixture (namely the cooled steam cracker effluent) at the primary fractionator inlet, etc. For example, the cooled steam cracker effluent can include the quench fluid in an amount of about 5 wt % to about 95 wt %, about 25 wt % to about 90 wt %, or about 50 wt %, or about 80 wt %, based on the weight of the cooled steam cracker effluent, i.e., the combined weight of the steam cracker effluent and the quench fluid.

In some examples, a steam cracker quench oil product separated from the steam cracker effluent can be recycled and contacted with the steam cracker effluent to produce the cooled steam cracker effluent. In some examples, in lieu of or in addition to using the steam cracker quench oil product to cool the steam cracker effluent, a steam cracker gas oil product and/or one or more utility fluid products can be used. Suitable utility fluid products can include those disclosed in U.S. Pat. Nos. 9,090,836:9,637,694; and 9,777,227; and International Patent Application Publication No. WO 2018/111574. Those skilled in the art will also appreciate that the amount of any contaminant-containing composition, i.e., acetic acid, carbon monoxide, and/or carbon dioxide contained in the quench fluid, if used, should be taken into account in determining the amount of contaminant-containing composition contained in the cooled steam cracker effluent.

The cooled steam cracker effluent can be introduced into one or more first separation stages, e.g., a tar knock out drum, to separate a tar product and a light product therefrom. In some examples, illustrative first separation stages can include those disclosed in U.S. Pat. Nos. 7,674,366; 7,718,049; 8,083,931; 8,092,671; 8,105,479.

The light product can be at a temperature of about 155° C., about 175° C., about 200° C., or about 225° C., to a about 250° C., about 270° C., about 290° C., about 300° C., or about 315° C. The tar product can be or can include, but is not limited to, a mixture of hydrocarbons having one or more aromatic components and, optionally, non-aromatic and/or non-hydrocarbon molecules, the mixture being derived from hydrocarbon pyrolysis, with at least 70% to about 100% of the mixture having a boiling point at atmospheric pressure that is at least 290° C., e.g., 290° C., to about 500° C. In some examples, the tar product can have an initial boiling point of at least 200° C., and/or a final atmospheric boiling point of >600° C., as measured according to ASTM D2887-18. In other examples, at least 90 wt % to about 100 wt % of the tar product can have a boiling point at atmospheric pressure at least 290° C., e.g., 290° C., to about 500° C. In some examples, the tar product that can be separated from the steam cracker effluent and processes for upgrading same can include those described in U.S. Patent Application Publication Nos.; 2010/00096296; 2015/0344785; 2015/0344790; 2016/0122667; 2018/0057759; 2018/0171239; 2019/0016969; and 2019/0016975.

The light product can be introduced into one or more second separation stages, e.g., a primary fractionator, to separate a steam cracker quench oil product, a steam cracker gas oil product, and the overhead therefrom. Steam cracker gas oil and steam cracker quench oil each include a mixture of compounds, primarily a mixture of hydrocarbon compounds. In some examples, at least a portion of the steam cracker quench oil product can be mixed, blended, combined, or otherwise contacted with the steam cracker effluent to produce the cooled steam cracker effluent. It should be understood that typically there is an overlap between pygas and steam cracker gas oil in composition and boiling point range. The final atmospheric boiling point of steam cracker gas oil is typically about 275° C.′ to about 285° C., as measured according to ASTM D2887-18. It should also be understood that typically there is an overlap between steam cracker gas oil and steam cracker quench oil in composition and boiling point range. The final atmospheric boiling point of steam cracker quench oil is typically about 455° C., to about 475° C., as measured according to ASTM D2887-18.

The overhead can include a process gas and pygas that can be introduced into one or more quench stages, e.g., a quench tower, and contacted with a quench medium, e.g., water or a recycled water, to cool the overhead and condense a mixture that includes water and pygas. The process gas and a naphtha cut can be recovered from the quench stage. The process gas can include, but is not limited to, hydrogen, methane, C₂ hydrocarbons, C₃ hydrocarbons, C₄ hydrocarbons, C₅₊ hydrocarbons, methanol, acetone, phenol, acetaldehyde, or any mixture thereof. The naphtha cut can include pygas, water, methanol, acetone, phenol, acetaldehyde, or any mixture thereof. Pygas, also referred to as steam cracker naphtha, is a complex mixture of C₅₊ hydrocarbons, e.g., C₅-C₁₀₊ hydrocarbons, having an initial atmospheric boiling point of about 25° C., to about 50° C., and a final boiling point of about 220° C., to about 265° C., as measured according to ASTM D2887-18. In some examples, pygas can have an initial atmospheric boiling point of about 33° C., to about 43° C., and a final atmospheric boiling point of about 234° C. to about 244° C., as measured according to ASTM D2887-18.

It should be understood that the first separation stage and the second separation stage, the second separation stage and the quench stage, or the first separation stage, the second separation stage, and the quench stage can be integrated with one another e.g., a single separation tower or column. In some examples, illustrative integrated separation stages can include those disclosed in U.S. Pat. Nos. 7,560,019; 8,105,479; and 8,197,668; and U.S. Patent Application Publication No 2014/0357923; and 2014/0376511.

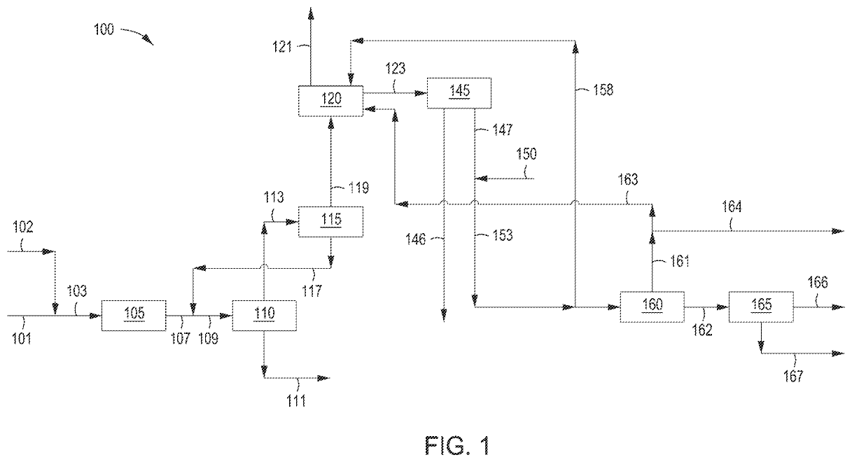

FIG. 1 depicts a schematic of an illustrative system 100 for steam cracking a hydrocarbon feed in line 101 to produce a steam cracker effluent via line 107 and accounting for or otherwise handling acetic acid contained therein, according to one or more embodiments. The system 100 can include one or more steam crackers 105, one or more first separation stages 110, e.g., tar knock-out drum, one or more second separation stages 115, e.g., primary fractionator, and one or more quench stages 120. The system 100 can optionally include one or more desalters (not shown) and/or one or more vapor/liquid separation stages (not shown) configured to separate a vapor phase product or first vapor phase product and a liquid phase product or first liquid phase product from a heated mixture of hydrocarbons and steam. The first vapor phase product can be introduced into a radiant section of the steam cracker 105 and the first liquid phase product can be further processed and/or used as fuel oil, for example. In some examples, the first separation stage 110, the second separation stage 115, and/or the quench stage 120 can be integrated with one another as described above. The system 100 can also include one or more third separation stages 145, one or more sour water strippers 160, and one or more dilution steam generators 165.

Prior to introducing the hydrocarbon feed in line 101 into the steam cracker 105, an amount of acetic acid that will be present in the steam cracker effluent in line 107 can be determined. As described above, the amount of acetic acid that will be present in the steam cracker effluent can be determined based, at least in part, on a composition of the hydrocarbon feed in line 101, a temperature the hydrocarbon feed will be heated at during steam cracking within the steam cracker 105, a residence time the hydrocarbon feed will be heated at the temperature during steam cracking, or a combination thereof.

It has been discovered that essentially all of the acetic acid present in the steam cracker effluent in line 107 goes with an overhead or light product separated via line 113 from a steam cracker effluent in line 107 in the first separation stage 110. It has also been discovered that essentially all of the acetic acid present in the overhead in line 113 goes with a naphtha cut or first naphtha cut via line 123 recovered from the quench stage 120. For example, ≥97 wt %. ≥99 wt %, ≥99.7 wt %, or ≥99.9 wt % of acetic acid present in the light product in line 113 can go with the first naphtha cut in line 123 recovered from the quench stage 120. It has also been discovered that essentially all of the acetic acid present in the first naphtha cut in line 123 goes with an aqueous mixture via line 147 recovered from the third separation stage 145. For example, ≥97 wt %, ≥99 wt %, ≥99.7 wt %, or ≥99.9 wt % of acetic acid present in the first naphtha cut in line 123 can go with the aqueous mixture in line 147 recovered from the third separation stage 145.

The aqueous mixture in line 147 can be contacted with a predetermined amount of a neutralizing agent in line 150 sufficient to neutralize the acetic acid in the first aqueous mixture, thereby producing treated mixture via line 153. Accordingly, prior to steam cracking the hydrocarbon feed in line 101, a sufficient amount of neutralizing agent, e.g., an aqueous mixture, having a predetermined concentration of the neutralizing agent can be prepared and made ready to be mixed, blended, or otherwise combined via line 150 with the aqueous mixture in line 147 at a predetermined flow rate based, at least in part, on the determined amount of acetic acid that will be present in the steam cracker effluent in line 107 to produce a treated mixture via line 153 that includes neutralized acetic acid. The pH of the aqueous mixture in line 147 can be <7.5, ≤7.0, ≤6.5, ≤6.0, ≤5.5; or ≤5.0. Preferably the pH is about 7.0.

The predetermined amount of neutralizing agent introduced via line 150 to the aqueous mixture in line 147 can be sufficient to neutralize ≥95 wt %, ≥97 wt %, ≥99 wt %, or ≥99.9 wt % of the acetic acid present in the aqueous mixture in line 147. The pH of the treated mixture in line 153 can be ≥6.0; ≥6.5; ≥7.0; 7.5; 8.0; ≥8.5; ≥9.0; or about 9.5. Preferably the pH in line 153 is about 7.0.

With the predetermined amount of neutralizing agent determined, the hydrocarbon in line 101 can be mixed, blended, or otherwise combined with steam in line 102 to produce a heated mixture via line 103 that can be introduced into the steam cracker 105 and subjected to steam cracking conditions to produce the steam cracker effluent via line 107, as described above. The hydrocarbon feed in line 101 can be any desired hydrocarbon feed, e.g., a crude oil or a fraction thereof. In some examples, the hydrocarbon feed in line 101 can be or can include a desalted crude oil derived from a raw crude oil. In some examples, the hydrocarbon feed in line 101 can be or can include a hydrocarbon feed having a total acid number of ≥0.5 mg KOH/g of hydrocarbon feed, as measured according to ASTM D664-18e2. In some examples, the mixture in line 103 or a vapor phase product separated therefrom can be steam cracked according to the processes disclosed in U.S. Pat. Nos. 6,419,885; 7,993,435; 9,637,694; and 9,777,227; and International Patent Application Publication No. WO 2018/111574.

The steam cracker effluent in line 107 can be contacted with a quench fluid, e.g., a steam cracker quench oil product via line 117, to produce a cooled steam cracker effluent in line 109. The cooled steam cracker effluent in line 109 can be introduced into the first separation stage 110 and a steam cracker tar product via line 111 and the overhead or light product via line 113 can be conducted away therefrom. The light product via line 113 can be introduced into the second separation stage 115 and the steam cracker quench oil product via line 117 and an overhead or first overhead via line 119 can be conducted away therefrom.

The first overhead via line 119 can be introduced into the quench stage 120 and can be contacted with a quench medium, e.g., water recovered from a downstream process such as water via line 158 and/or 163, to produce a cooled or quenched overhead. An overhead or second overhead via line 121 and the first naphtha cut via line 123 can be conducted away from the quench stage 120. The second overhead in line 121 can include, but is not limited to, hydrogen, methane, C₂ hydrocarbons, C₃ hydrocarbons, C₄ hydrocarbons, C₅ hydrocarbons, or any mixture thereof. In some examples, the second overhead in line 121 can include a first portion of the acetic acid, e.g., ≤3 wt %, ≤1 wt %, ≤0.3 wt %, or ≤0.1 wt % of the acetic acid present in the steam cracker effluent in line 107. In some examples, the second overhead in line 121 can be free of any acetic acid. The first naphtha cut in line 123 can include, but is not limited to, a second portion of the acetic acid, pygas, and quench medium, e.g., water. In some examples, the first naphtha cut in line 123 can include ≥97 wt %, ≥99 wt %, ≥99.7 wt %, ≥99.9 wt %, or 100% of the acetic acid present in the first overhead in 119.

The second overhead in line 121 can further processed within one or more process gas upgrading stages (not shown) according to well-known processes. A number of products can be separated from the second overhead in line 121. For example, hydrogen, ethylene, ethane, propylene, propane, butene-1, a raffinate, diisobutylene, and/or other products can be separated from the second overhead in line 121.

The first naphtha cut via line 123 can be introduced into the third separation stage 145 to produce a pygas product via line 146 and an aqueous mixture via line 147 that can include acetic acid, e.g., ≥97 wt % of the acetic acid present in the first naphtha cut in line 123. The predetermined amount of neutralizing agent via line 150 can be mixed, blended, or otherwise combined with the aqueous mixture in line 147 to produce the treated mixture in line 153 that includes neutralized acetic acid. The predetermined amount of neutralizing agent via line 150 can be sufficient to neutralize ≥90 wt %, ≥95 wt %, ≥97 wt %, ≥99 wt %, ≥99.5 wt %, ≥99.9 wt % of the acetic acid present in the aqueous mixture in line 147. The neutralizing agent can be or can include any compound or mixture of compounds capable of neutralizing the acetic acid present in the first naphtha cut in line 123. Suitable neutralizing agents can be or can include, ammonia, organic amines such as primary amines (R—NH₂, e.g., monoethanol amine), secondary amines (R¹—NH—R²), tertiary amines (R¹R²—N—R³), and diamines, caustics such as NaOH and KOH, or any mixture thereof.

The treated mixture via line 153 can be introduced into the sour water stripper 160 to produce a sour water stripper overhead or third overhead via line 161 and a sour water stripper naphtha cut or a second bottoms via line 162. In some examples, a portion of the treated mixture in line 153 can be recycled via line 158 to the quench stage 120 as at least a portion of the quench medium introduced thereto. In some examples, the aqueous mixture can be contacted with steam, e.g., counter currently, within the sour water stripper 160, to produce the third overhead via line 161. The third overhead in line 161 can include steam and hydrogen sulfide, ammonia, or a mixture hydrogen sulfide and ammonia.

In some examples, the third overhead in line 161 can include a first portion of the neutralized acetic acid and the second bottoms in line 162 can include a second portion of the neutralized acetic acid. In other examples, the third overhead in line 161 can be free of any neutralized acetic acid and the second bottoms in line 162 can include all the neutralized acetic acid contained in the treated mixture in line 153. The third overhead in line 161 can include ≤ 3 wt %. ≤2 wt %, ≤ 1 wt %, ≤ 0.5 wt %, ≤0.3 wt %, or ≤ 0.1 wt % of the neutralized acetic acid contained in the treated mixture in line 153. In some examples, the second bottoms in line 162 can include ≥97 wt %. ≥99 wt %, ≥99.7 wt %. ≥99.9 wt %, or 100% of the neutralized acetic acid present in the treated mixture in line 153.

In some examples, at least a portion of the second vapor phase product in line 161 can be condensed, e.g., via indirect heat exchange or direct contact with a cooling medium such as water, to produce a process water or overhead condensate via line 164. In some examples, a portion of the third overhead in line 161 can be recycled via line 163 to the quench stage 120 as at least a portion of the quench medium introduced thereto.

In some examples, a mass flow rate of the overhead condensate in line 164 can be about 1%, about 5%, about 10%, about 20%, about 30%, or about 40% to about 60%, about 70%, about 80%, about 90%, or about 100% of a mass flow rate of the third overhead in line 161. In other examples, the mass flow rate of the overhead condensate in line 164 can be about 1% to about 20%, about 3% to about 15, about 5% to about 20%, about 10% to about 35%, about 5% to about 15%, about 25% to about 50%, about 40% to about 80%, or about 50% to about 90% of the mass flow rate of the third overhead in line 161.

In some examples, a mass flow rate of the overhead condensate in line 164 can be about 1%, about 3%, about 5%, or about 7% to about 10%, about 12%, about 15%, about 17%, or about 20% of a mass flow rate the aqueous mixture in line 147 separated from the first naphtha cut in line 123. In other examples, the mass flow rate of the overhead condensate in line 164 can be about 1% to about 20%, about 3% to about 15%, about 5% to about 10% about 4% to about 12%, or about 3% to about 15% the mass flow rate the aqueous mixture in line 147 separated from the first naphtha cut in line 123.

The second bottoms via line 162 can be conducted away from the sour water stripper 160 and can be introduced into the dilution steam generator 165. The dilution steam generator 165 can heat the second bottoms in line 162 to produce dilution steam. The dilution steam via line 166 and a bottoms or third bottoms via line 167 be conducted away from the dilution steam generator 165. The third bottoms in line 167 can include ≥97 wt %, ≥98 wt %. ≥99 wt %, ≥ 99.5 wt %, or ≥99.9 wt % of the neutralized acetic acid present in the second bottoms in line 162. The third bottoms in line 167 can include ≥97 wt %. ≥98 wt %. ≥99 wt %, ≥99.5 wt %, or ≥99.9 wt % of the neutralized acetic acid present in the treated mixture in line 153. In some examples, the third bottoms in line 167 can include ≥95 wt %, ≥97 wt %. ≥98 wt %, ≥99 wt %. ≥99.5 wt %, or ≥99.9 wt % of the acetic acid present in the steam cracker effluent in line 107, in the neutralized form. The third bottoms via line 167 can be removed from the process as a process water.

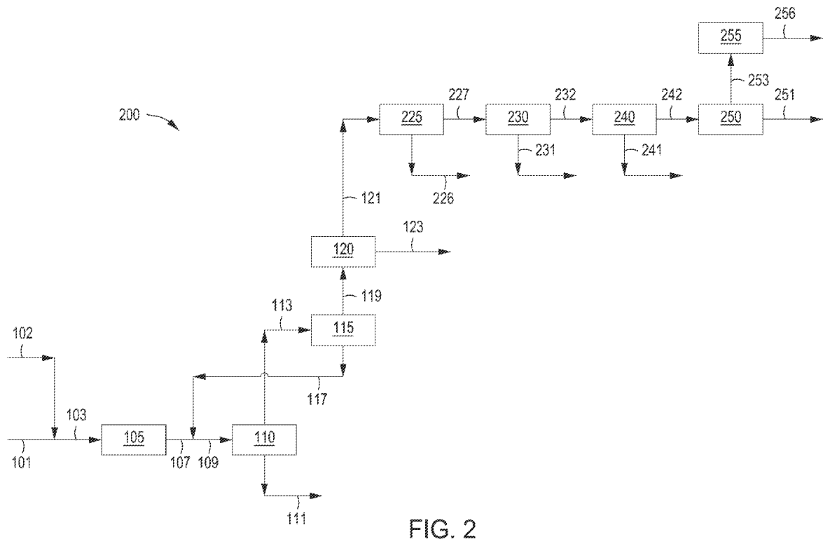

FIG. 2 depicts a schematic of an illustrative system 200 for steam cracking a hydrocarbon feed in line 101 to produce a steam cracker effluent via line 107 and accounting for or otherwise handling carbon monoxide contained therein, according to one or more embodiments. The system 200 can include the one or more steam crackers 105, the one or more first separation stages 110, the one or more second separation stages 115, and the one or more quench stages 120, as described above with reference to FIG. 1. The system 200 can also include one or more process gas upgrading stages 225, one or more depropanizers 230, one or more demethanizers 240, one or more gas separation stages or “cold boxes” 250, and one or more hydrogen-rich gas upgrading stages 255.

The overhead or second overhead via line 121 and a naphtha cut or first naphtha cut via line 123 can be recovered from the quench stage 120 as described above with reference to FIG. 1. The second overhead in line 121 can include, but is not limited to, carbon monoxide, hydrogen, methane, C₂ hydrocarbons, C₃ hydrocarbons, C₄ hydrocarbons, C₅ hydrocarbons, or any mixture thereof. The first naphtha cut in line 123 can include, but is not limited to, pygas, and a quench medium, e.g., water.

The hydrocarbon feed in line 101 typically does not include carbon monoxide, which is primarily if not exclusively produced during steam cracking of the hydrocarbon feed in the steam cracker 105. In some examples, an amount of carbon monoxide that will be present in the steam cracker effluent in line 107 can be based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof.

It has been discovered that steam cracking a representative crude oil feed or a fraction thereof (e.g., an overhead stream from a flashing drum separating a crude oil feed) in line 101 can produce a steam cracker effluent comprising, based on the total weight of the steam cracker effluent, from 50, 60, 70, 80, 90 ppm by weight, to 100, 200, 300, 400, 500 ppm by weight, to 600, 700, 800, 900, 1,000 ppm by weight, to 1.500, 2,000, 2,500, 3,000, 3,500 ppm by weight, to 4.000, 4,500, or 5,000 ppm by weight, of CO. In some examples, at least a portion of the carbon monoxide can be produced by decomposing methanol and/or naphthenic acids present in the hydrocarbon feed in line 101 and/or during online decoking operations of the steam cracker furnace. Should the hydrocarbon feed be contaminated with carbon monoxide, any carbon monoxide present in the hydrocarbon feed can be readily determined by determining the composition of the hydrocarbon feed in line 101.

The second overhead in line 121 can include ≥97 wt %, ≥98 wt %, ≥99 wt %. ≥99.5 wt %, ≥99.9 wt %, or 100 wt % of the carbon monoxide present in the steam cracker effluent in line 107. The second overhead via line 121 can be subjected to an amine and/or caustic treatment within the process gas upgrading stage 225 to produce a process water or first process water via line 226 and an upgraded second overhead via line 227. The upgraded second overhead in line 227 can include ≥97 wt %. ≥98 wt %, ≥99 wt %, ≥99.5 wt %. ≥99.9 wt %, or 100 wt % of the carbon monoxide present in the steam cracker effluent in line 107.

A hydrogen-rich gas via line 253 and a fuel gas via line 251 can be separated from the upgraded second overhead in line 227. In some examples, the upgraded second overhead via line 227 can be introduced into the depropanizer 230 to produce a depropanizer bottoms via line 231 and a depropanizer overhead via line 232. The depropanizer overhead via line 232 can be introduced into the demethanizer 240 to produce a demethanizer bottoms via line 241 and a demethanizer overhead via line 242. The demethanizer overhead in line 242 can include ≥97 wt %, ≥98 wt %, ≥99 wt %, ≥99.5 wt %, ≥99.9 wt %, or 100 wt % of the carbon monoxide present in the steam cracker effluent in line 107. While not shown, in some examples, the depropanizer overhead can optionally be treated to remove or reduce a concentration of one or more impurities that can be present. For example, the depropanizer overhead can be treated to remove at least a portion of any carbonyl sulfide, arsine, and/or acetylene. The demethanizer overhead via line 242 can be introduced into the cold box 250 to separate the fuel gas via line 251 and the hydrogen-rich gas via line 253 therefrom.

The hydrogen-rich gas in line 253 can include a first portion of the carbon monoxide contained in the upgraded second overhead in line 227, molecular hydrogen, methane, or a mixture thereof. The fuel gas in line 251 can include a second portion of the carbon monoxide contained in the upgraded second overhead in line 227, CH₄, H₂, CO, ethylene, and trace contaminants if present, e.g., O₂, N₂, NOx, etc.

For a wide range of conventional crude oil feeds, the cold box 250 can be operated under conditions sufficient to cause ≥40 wt %, ≥50 wt %. ≥60 wt %, ≥70 wt %, up to about 80 wt % of the carbon monoxide in the demethanizer overhead in line 242 to exit with the hydrogen-rich gas via line 253, with the remainder exiting with the fuel gas line 251.

The hydrogen-rich gas in line 253 can include, e.g., about 0 wt % to about 44 wt % of carbon monoxide, about 40 wt % to about 100 wt % of molecular hydrogen, and about 0 wt % to about 50 wt % of methane. The fuel gas in line 251 can include, e.g., about 0 wt % to about 30 wt % of carbon monoxide, about 0 wt % to about 30 wt % of H2, and about 70 wt % to about 100 wt % of methane.

The hydrogen-rich gas via line 253 can be introduced into the hydrogen-rich gas upgrading stages 255 to produce a purified hydrogen gas product via line 256. In some examples, the hydrogen-rich gas upgrading stage 255 can be or can include one or more methanators, one or more pressure swing adsorption units, or a combination thereof.

If the hydrogen-rich gas upgrading stage 255 includes a methanator, the methanator can have a predetermined size sufficient to convert a majority, preferably a great majority, e.g. ≥90 wt %, ≥92 wt %, ≥94 wt %, ≥95 wt %, ≥97 wt %, ≥99 wt %, ≥99.5 wt %, ≥99.9 wt %, or ≥99.95 wt % of the first portion of carbon monoxide to methane, thereby abating the carbon monoxide in the hydrogen-rich gas. The predetermined size of the methanator can be based, at least in part on the determined amount of carbon monoxide that will be present in the steam cracker effluent in line 107. The methanator can have a predetermined size sufficient to maintain a heat of reaction within the methanator. The methanator can include one or more catalysts disposed therein. The catalyst can facilitate the conversion of carbon monoxide to methane. Suitable catalysts can be or can include, but are not limited to, catalysts that include nickel, rhodium, ruthenium, or a mixture thereof.

If the hydrogen-rich gas upgrading stage 255 includes a pressure swing adsorption unit, the pressure swing adsorption unit can have a predetermined size sufficient to remove a majority, preferably a great majority, e.g., ≥90 wt %, ≥92 wt %, ≥94 wt %, ≥95 wt %, ≥97 wt %, ≥99 wt %. ≥99.5 wt %, ≥99.9 wt %, or ≥99.95 wt % of the first portion of carbon monoxide, thereby abating the carbon monoxide in the hydrogen-rich gas. The predetermined size of the pressure swing adsorption unit can be based, at least in part on the determined amount of carbon monoxide that will be present in the steam cracker effluent in line 107.

The purified hydrogen gas product recovered via line 256, when the hydrogen-rich gas upgrading stage 255 includes a methanator and/or a pressure swing adsorption unit can include ≤5 wt %, ≤3 wt %, ≤1 wt %, ≤0.5 wt %, ≤0.1 wt %, or ≤0.05 wt % of carbon monoxide. In some examples, the purified hydrogen gas product recovered via line 256, when the hydrogen-rich gas upgrading stage 255 includes a methanator can include ≤5 wt %, ≤3 wt %, ≤1 wt %, ≤0.5 wt %, ≤0.1 wt %, or ≤0.05 wt % of the carbon monoxide contained in the steam cracker effluent in line 107. Typically, it is highly desirable that substantially all of the CO is removed from the hydrogen-rich gas, such that the effluent exiting the methanator and/or the pressure swing adsorption unit comprises CO at an exceedingly low concentration, e.g., <1 ppm by weight, based on the total weight of the effluent exiting the methanator and/or the pressure swing adsorption unit.

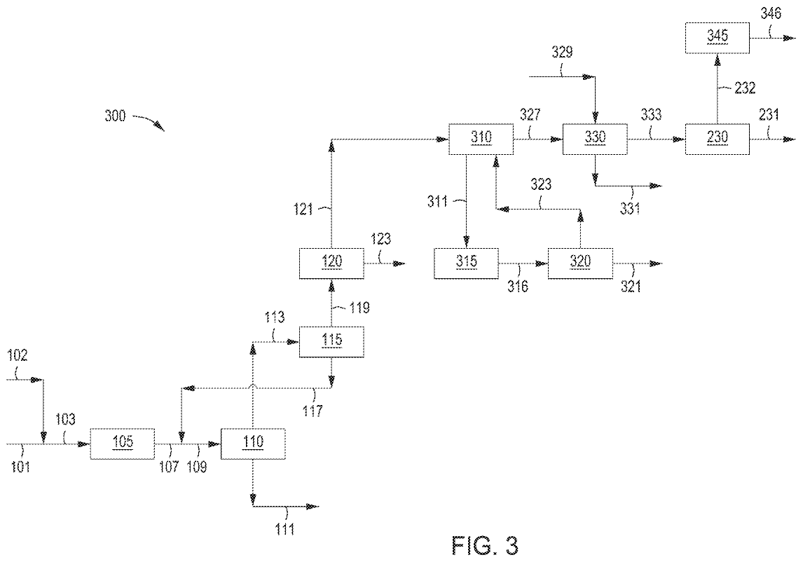

FIG. 3 depicts a schematic of an illustrative system 300 for steam cracking a hydrocarbon feed in line 101 to produce a steam cracker effluent via line 107 and accounting for or otherwise handling carbon dioxide contained therein, according to one or more embodiments. The system 300 can include the one or more steam crackers 105, the one or more first separation stages 110, e.g., tar knock-out drum, the one or more second separation stages 115, e.g., primary fractionator, the one or more quench stages 120, and the one or more depropanizers 230, as described above with reference to FIGS. 1 and/or 2. The system 300 can also include one or more amine units 310, one or more amine regeneration units 315, one or more lean amine storage units 320, one or more caustic units 330, one or more depropanizers 230, and one or more carbonyl sulfide removal units 345.

The hydrocarbon feed in line 101 typically does not include carbon dioxide, which is primarily produced during steam cracking of the hydrocarbon feed in the steam cracker 105. In some examples, an amount of carbon dioxide that will be present in the steam cracker effluent in line 107 can be based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof. In some examples, at least a portion of the carbon dioxide can be produced by decomposing naphthenic acids present in the hydrocarbon feed in line 101, cracking of hydrocarbons in the hydrocarbon feed, and/or during online decoking operations of the steam cracker furnace. Should the hydrocarbon feed be contaminated with carbon dioxide, any carbon dioxide present in the hydrocarbon feed can be readily determined by determining the composition of the hydrocarbon feed in line 101. It has been discovered that steam cracking the hydrocarbon feed can produce a steam cracker effluent comprising CO₂ at a concentration from 50, 60, 70, 80, 90 ppm by weight, to 100, 200, 300, 400, 500 ppm by weight, to 600, 700, 800, 900, 1,000 ppm by weight, to 1,500, 2,000, 2,500, 3,000, 3,500 ppm by weight, to 4,000, 4,500, or 5,000 ppm by weight, based on the total weight of the steam cracker effluent.

In some examples, the amine unit 310 can have a predetermined capacity or size, which can be evaluated and/or pre-determined based on steam cracking conditions used to crack the hydrocarbon feed in line 101. The amine unit 310 can be adapted or configured to produce an amine treated second overhead or a third overhead via line 327 and a spent amine or second bottoms via line 311. The amine unit 310 can remove carbon dioxide from the second overhead in line 121. The spent amine can include a reaction product of the amine and carbon dioxide. The predetermined capacity of the amine unit 310 can be based, at least in part, on the determined amount of carbon dioxide that will be present in the steam cracker effluent in line 107. Any suitable amine or mixture of amines can be utilized in the amine unit 310. Suitable amines can be or can include, but are not limited to, monoethanol amine, diethanol amine, triethanol amine, diglycol amine, diisoproanalamine, methy diethylanolamine, or any mixture thereof, although using methy diethylanolamine may lead to a need for removing additional CO₂ at locations downstream of the amine unit, e.g., in a caustic tower.

The amine regeneration unit 315 can be adapted or configured to regenerate the spent amine introduced via line 310 thereto. The predetermined capacity of the amine regeneration unit 315 can be based, at least in part, on the determined amount of carbon dioxide that will be present in the steam cracker effluent in line 107. The regenerated amine or lean amine via line 316 can be introduced into the lean amine storage 320 and recycled as needed to the amine unit 310. A water purge via line 321 can be conducted away from the lean amine storage as needed to maintain a predetermined concentration of the amine in the lean amine recycled via line 323 to the amine unit 310.

In other examples, an aqueous amine solution having a predetermined concentration of amine can be prepared. The predetermined concentration of the amine in the aqueous amine solution can be based, at least in part, on the determined amount of carbon dioxide that will be present in the steam cracker effluent in line 107. If the aqueous amine solution having the predetermined concentration is prepared, the aqueous amine solution in line 323 can have the predetermined concentration of the amine and can be introduced via line 323 into the amine unit 310 to contact the second overhead introduced via line 121 to produce a treated mixture therein. To suit the needs of abating differing amounts of CO₂ in the stream cracker effluents producing from different feeds at various feeding rate under various steam cracking conditions, the concentration of the pre-prepared aqueous amine solution may be diluted or increased (by, e.g., blending with amine solution having a higher concentration) as needed. The flow rate of the stream in line 323 can be also adjusted to suit the needs of abating differing amounts of CO₂ in the stream cracker effluent(s). The aqueous amine solution in line 323 can have an amine concentration ranging from, e.g., 5, 6, 7, 8, 9 wt %, to 12, 14, 15, 16, 18, 20 wt %, to 22, 24, 25, 26, 28, 30 wt %, to 32, 34, 35, 36, 38, 40 wt %, to 42, 44, 45, 46, 48, 50 wt %, to 52, 54, 55, 56, 58, 60 wt %, based on the total weight of the stream in line 323. Desirably, the molar amount of amine fed into the amine unit 310 is higher than the molar amount of CO₂ and H₂S to be abated therein. For example, the molar ratio of the amine fed into amine unit 310 to the total of CO₂ and H₂S can desirably vary from, e.g., 1.5, 1.6, 1.8, 2.0 to 2.2, 2.4, 2.5, 2.6, 2.8, 3.0, to 3.2, 3.4, 3.5, 3.6, 3.8, 4.0, to 4.2, 4.4, 4.5, 4.6, 4.8, 5.0.

The amine unit 310 having the predetermined size and/or the aqueous amine solution having the predetermined concentration of amine can be sufficient to remove ≥75 wt %, ≥80 wt %. ≥85 wt %. ≥87 wt %, or ≥90 wt % of the carbon dioxide contained in the second overhead in line 121 by producing the spent amine or second bottoms via line 311 that can include the reaction product of the amine and carbon dioxide. In some examples, the predetermined size of the amine unit 310 and/or the concentration of the amine in the aqueous amine solution having the predetermined concentration of amine can be sufficient to remove about 75 wt %, about 80 wt %, or about 85 wt % to about 90 wt %, about 93 wt %, or about 95 wt % of the carbon dioxide contained in the steam cracker effluent in line 107. In some examples, the predetermined size of the amine unit 310 and/or the concentration of the amine in the aqueous amine solution having the predetermined concentration of amine can be sufficient to remove about 75 wt %, about 80 wt %, or about 85 wt % to about 90 wt %, about 93 wt %, or about 95 wt %, and up to 99.9 wt % of the carbon dioxide contained in the second overhead in line 121.

In some examples, the caustic unit 330, which can have a predetermined capacity, can be installed prior to steam cracking the hydrocarbon feed in line 101. The caustic unit 330 can be adapted or configured to produce a fourth overhead via line 333 by removing carbon dioxide from the third overhead in line 327. In some examples, an aqueous caustic solution via line 329 can be introduced into the caustic unit 330 and can contact the third overhead introduced via line 327 to produce a spent caustic or third bottoms via line 331, which can include a reaction product of the caustic and carbon dioxide and a fourth overhead via line 333. The predetermined capacity of the caustic unit 330 can be based, at least in part, on the determined amount of carbon dioxide that will be present in the steam cracker effluent in line 107. Any suitable caustic or mixture of caustic compounds can be utilized in the amine unit 310. Suitable caustics can be or can include, but are not limited to, sodium hydroxide, potassium hydroxide, etc., or any mixture thereof.

In other examples, an aqueous caustic solution in line 329 having a predetermined concentration of caustic can be prepared. The predetermined concentration of the caustic in the aqueous caustic solution can be based, at least in part, on the determined amount of carbon dioxide that will be present in the steam cracker effluent in line 107. If the aqueous caustic solution in line 329 has the predetermined concentration of caustic, the aqueous caustic solution in line 329 can comprise the caustic at a concentration ranging from, e.g., 2, 4, 5, 6, 8, 10 wt %, to 12, 14, 15, 16, 18, 20 wt %, to 22, 24, 25, 26, 28, 30 wt %, to 32, 34, 35, 36, 38, 40 wt %, to 42, 44, 45, 46, 48, 50 wt %, based on the total weight of the aqueous caustic solution.

The caustic unit 330 having the predetermined size and/or the aqueous caustic solution in line 329 having the predetermined concentration can be sufficient to remove ≥75 wt %, ≥80 wt %, ≥85 wt %, ≥87 wt %, or ≥90 wt % of the carbon dioxide contained in the third overhead in line 327 by producing the spent caustic that can include the reaction product of the caustic and carbon dioxide. In some examples, the predetermined size of the caustic unit 330 and/or the aqueous caustic solution in line 329 having the predetermined concentration can be sufficient to remove about 75 wt %, about 80 wt %, or about 85 wt % to about 90 wt %, about 93 wt %, or about 95 wt % of the carbon dioxide contained in the third overhead in line 327. In some examples, the fourth overhead in line 333 can contain ≤7 wt %, ≤5 wt %, ≤3 wt %. ≤1 wt %, ≤0.5 wt %, ≤0.1 wt %, ≤0.05 wt %, or ≤0.01 wt % of the carbon dioxide contained in the steam cracker effluent in line 107. In some examples, the fourth overhead in line 333 can contain ≤7 wt %, ≤5 wt %, ≤3 wt %, ≤2.5 wt %, ≤1 wt %. ≤0.5 wt %, ≤0.1 wt %, ≤0.05 wt %, or ≤0.01 wt % of the carbon dioxide contained in the second overhead in line 121.

It should be understood that the amine unit 310 and the caustic unit 330 can be switched with one another such that the first overhead in line 121 can be introduced into the caustic unit 330 to produce the third overhead in line 327 and the third overhead in line 327 introduced into the amine unit 310 to produce fourth overhead via line 333. Preferably the amine unit 310 is located upstream of the caustic unit 330.

With the amine unit 310 and the amine regeneration unit 315 and/or the caustic unit 330 installed and/or the aqueous amine solution having the predetermined concentration of amine in line 323 and/or the aqueous caustic solution having the predetermined concentration of caustic in line 329 prepared, the hydrocarbon feed in 101 can be mixed, blended, or otherwise combined with steam in line 102 to produce a heated mixture via line 103 that can be introduced into the steam cracker 105 and subjected to steam cracking conditions to produce a steam cracker effluent via line 107, as described above. The second overhead via line 121 and the first bottoms via line 123 can be recovered from the quench stage 120 as described above with reference to FIGS. 1 and 2. The second overhead in line 121 can include, but is not limited to, carbon dioxide, hydrogen, methane. C₂ hydrocarbons. C; hydrocarbons, C₄ hydrocarbons, C₅ hydrocarbons, or any mixture thereof. The first bottoms in line 123 can include, but is not limited to, pygas, and a quench medium, e.g., water.

The second overhead in line 121 can include ≥97 wt %. ≥98 wt %, ≥99 wt %, ≥99.5 wt %. ≥99.9 wt %, or 100 wt % of the carbon dioxide present in the steam cracker effluent in line 107. In some examples, the second overhead via line 121 can be subjected to an amine and/or caustic treatment within the amine unit 310 and/or the caustic unit 330 to produce the third overhead via line 327 and the fourth overhead via line 333, respectively.

The amine unit 310 having the predetermined size and/or the aqueous amine solution having the predetermined concentration of amine can remove ≥75 wt %, ≥80 wt %, ≥85 wt %, ≥87 wt %, or ≥90 wt % of the carbon dioxide contained in the second overhead in line 121 by producing the spent amine or second bottoms via line 311 that can include the reaction product of the amine and carbon dioxide. In some examples, the third overhead in line 327 can contain ≤25 wt %, ≤20 wt %. ≤15 wt %, ≤13 wt %, or ≤10 wt % of the carbon dioxide contained in the steam cracker effluent in line 107. In some examples, the third overhead in line 327 can contain ≤25 wt %, ≤ 20 wt %. ≤ 15 wt %, ≤ 13 wt %, or ≤10 wt % of the carbon dioxide contained in the second overhead in line 121.

The process conditions within the amine unit 310 can include a gauge pressure from e.g., 1,000, 1,100, 1,200, 1,300, 1,400, 1,500 kPa to 1,600, 1,700, 1,800, 1,900, 2,000 kPa; and a temperature from, e.g., 20, 22, 24, 25, 26, 28, 30° C., to 32, 34, 35, 36, 38, 40° C., to 42, 44, 45, 46, 48, 50° C., to 52, 54, 55, 56, 58, 60° C. The process conditions within the amine regeneration unit 315 can include a gauge pressure from, e.g., 50, 60, 70, 80, 90, 100 kPa, to 120, 140, 150, 160, 180, 200, to 220, 240, 250, 260, 280, 300 kPa, and a temperature from, e.g. 100, 110, 120, 130, 140, 150° C., to 160, 170, 180, 190, 200° C., depending on factors such as the type of amine used.

The third overhead via line 327 can be introduced into the caustic unit 330 to produce the fourth overhead via line 333 and the spent caustic or third bottoms via line 331. In some examples, the fourth overhead in line 333 can contain ≤0 wt %, ≤15 wt %, ≤13 wt %, or ≤10 wt % of the carbon dioxide contained in the steam cracker effluent in line 107. In some examples, the third overhead in line 327 can contain ≤25 wt %, ≤20 wt %. ≤15 wt %. ≤13 wt %, or ≤10 wt % of the carbon dioxide contained in the second overhead in line 121.

The process conditions within the caustic unit 330 can include a gauge pressure from e.g., 500, 600, 700, 800, 900, 1,000 kPa, to 1,100, 1,200, 1,300, 1,400, 1,500 kPa, to 1,600, 1,700, 1,800, 1,900, 2,000 kPa; and a temperature from, e.g., 20, 22, 24, 25, 26, 28, 30° C., to 32, 34, 35, 36, 38, 40° C., to 42, 44, 45, 46, 48, 50° C., to 52, 54, 55, 56, 58, 60° C.

In some examples, the fourth effluent in line 333 can be introduced into the depropanizer 230 to produce the depropanizer bottoms via line 231 and the depropanizer overhead via line 232. If the fourth effluent in line includes any carbon dioxide, ≥95 wt %, ≥ 97 wt %. ≥99 wt %. ≥99.9 wt % of the carbon dioxide can exit the depropanizer 231 as a component of the depropanizer overhead via line 232.

In some examples, a sufficient or predetermined amount of a sorbent can be introduced into the carbonyl sulfide removal unit 345 to allow the carbonyl sulfide removal unit to process the depropanizer overhead in line 232 for at least as long as the predetermined period of time without requiring replacement or re-activation of the sorbent due to deactivation caused by carbon dioxide present in the depropanizer overhead in line 232. The predetermined amount of sorbent can be based, at least in part, on the determined amount of carbon dioxide that will be present in the steam cracker effluent in line 107. During contact between the depropanizer overhead and the sorbent, the sorbent can remove at least a portion of the carbon dioxide present in the depropanizer overhead and a treated depropanizer overhead via line 136 can be conducted away from the carbonyl sulfide removal unit 345. For example, the sorbent can adsorb at least a portion of the carbon dioxide present in the depropanizer overhead to produce the treated depropanizer overhead in line 346. As such, the treated depropanizer overhead in line 346 can include less carbon dioxide than the depropanizer overhead in line 232.

The treated depropanizer overhead in line 346 can include, but is not limited to, molecular hydrogen, methane, ethylene, ethane, propylene, propane, or any mixture thereof. In some examples, the treated depropanizer overhead in line 346 can include carbon dioxide, but the amount of carbon dioxide present in the treated depropanizer overhead in line 346 can be ≤1 wt %. ≤0.7 wt %, ≤0.5 wt %, or ≤0.1 wt % of the carbon dioxide present in the depropanizer overhead in line 232. In other examples, the treated depropanizer overhead in line 346 can be free of any carbon dioxide.

The sorbent can be or can include one or more adsorbent materials, absorbent materials, a mixture thereof, or a combination thereof. In some examples, the sorbent can be or can include at least one metal from Group 1, 10, or 11 of the periodic table of elements or an oxide thereof. A non-limiting example of a useful sorbent material is Selexsorb available from BASF.

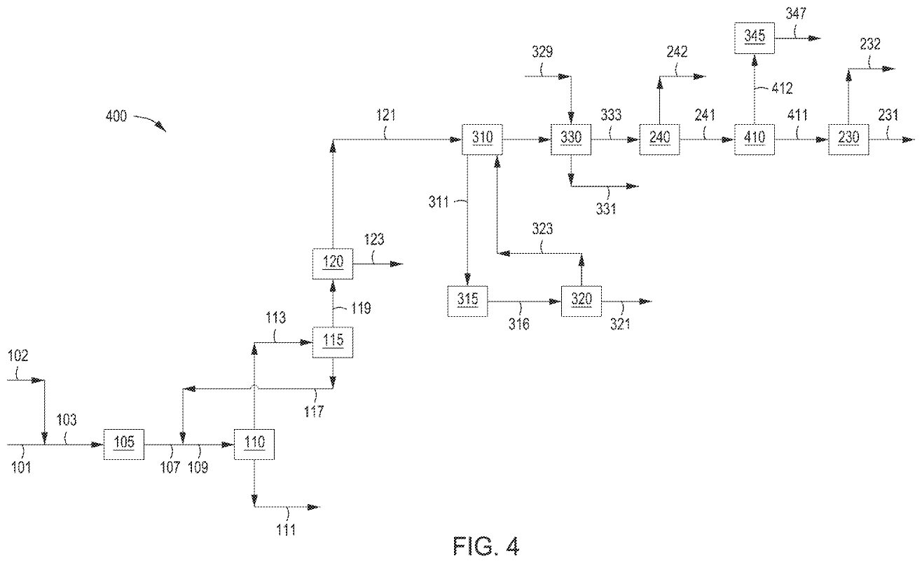

FIG. 4 depicts a schematic of another illustrative system 400 for steam cracking a hydrocarbon feed in line 101 to produce a steam cracker effluent via line 107 and accounting for or otherwise handling carbon dioxide contained therein, according to one or more embodiments. The system 300 can include the one or more steam crackers 105, the one or more first separation stages 110, e.g., tar knock-out drum, the one or more second separation stages 115, e.g., primary fractionator, the one or more quench stages 120, the one or more depropanizers 230, the one or more demethanizers 240, the one or more amine units 310, the one or more mine regeneration units 315, the one or more lean amine storage units 320, the one or more caustic units 330, and the one or more carbonyl sulfide removal units 345, as described above with reference to FIGS. 1 and/or 2. The system 400 can also include one or more deethanizers 410.

The system 400 can steam crack the hydrocarbon feed in line 101 to produce the steam cracker effluent via line 107 and process the steam cracker effluent to produce the fourth overhead via line 333 as described above with reference to FIGS. 1, 2, and/or 3. In some examples, the fourth overhead in line 333 can contain ≤7 wt %, ≤5 wt %, ≤3 wt %, ≤1 wt %, ≤ 0.5 wt %, ≤0.1 wt %. ≤0.05 wt %, or ≤0.01 wt % of the carbon dioxide contained in the steam cracker effluent in line 107. In some examples, the fourth overhead in line 333 can contain ≤ 7 wt %, ≤5 wt %, ≤3 wt %, ≤2.5 wt %, ≤1 wt %, ≤0.5 wt %, ≤0.1 wt %, ≤0.05 wt %, or ≤0.01 wt % of the carbon dioxide contained in the second overhead in line 121.

Rather than introducing the fourth overhead via line 333 into the depropanizer 230, as shown in FIG. 3, the fourth overhead via line 333 can be introduced into the demethanizer 240 to produce the demethanizer bottoms via line 241 and the demethanizer overhead via line 242. In some examples, the demethanizer overhead in line 242 can be further processed as described above with reference to FIG. 2.

When the fourth overhead in line 333 includes carbon dioxide, the carbon dioxide can exit the demethanizer 240 as a component of the demethanizer bottoms via line 241. The demethanizer bottoms via line 241 can be introduced into the deethanizer 410 to produce a deethanizer bottoms via line 411 and a deethanizer overhead via line 412. When the fourth overhead in line 333 includes carbon dioxide, the carbon dioxide can exit the deethanizer 410 as a component of the deethanizer overhead via line 412. When the fourth effluent in line 333 includes carbon dioxide, ≥95 wt %, ≥97 wt %. ≥99 wt %, ≥99.9 wt % of the carbon dioxide can exit the deethanizer 410 as a component of the deethanizer overhead via line 412. As such, in some examples, the deethanizer overhead in line 412 can contain ≤7 wt %. ≤5 wt %. ≤3 wt %, ≤ 1 wt %. ≤0.5 wt %, ≤ 0.1 wt %. ≤ 0.05 wt %, or ≤ 0.01 wt % of the carbon dioxide contained in the steam cracker effluent in line 107. Likewise, in some examples, the deethanizer overhead in line 412 can contain ≤7 wt %, ≤5 wt %, ≤3 wt %, ≤2.5 wt %, ≤1 wt %, ≤0.5 wt %, ≤0.1 wt %, ≤0.05 wt %, or ≤0.01 wt % of the carbon dioxide contained in the second overhead in line 121.

In some examples, a sufficient or predetermined amount of a sorbent can be introduced into the carbonyl sulfide removal unit 345 to allow the carbonyl sulfide removal unit to process the deethanizer overhead in line 412 for at least as long as the predetermined period of time without requiring replacement or re-activation of the sorbent due to deactivation caused by carbon dioxide present in the deethanizer overhead in line 412. The predetermined amount of sorbent can be based, at least in part, on the determined amount of carbon dioxide that will be present in the steam cracker effluent in line 107. In some examples, the predetermined amount of sorbent can be based, at least in part, on the capacity of the amine unit 310 and/or the caustic unit 330 to remove carbon dioxide from the second overhead in line 121. During contact between the deethanizer overhead and the sorbent, the sorbent can remove at least a portion of the carbon dioxide present in the depropanizer overhead and a treated deethanizer overhead via line 347 can be conducted away from the carbonyl sulfide removal unit 345. For example, the sorbent can adsorb at least a portion of the carbon dioxide present in the deethanizer overhead in line 412 to produce the treated deethanizer overhead in line 347. As such, the treated deethanizer overhead in line 347 can include less carbon dioxide than the deethanizer overhead in line 412.

The treated deethanizer overhead in line 347 can include, but is not limited to, ethylene, ethane, or a mixture thereof. In some examples, the treated deethanizer overhead in line 347 can include carbon dioxide, but the amount of carbon dioxide present in the treated deethanizer overhead in line 347 can be ≤5 wt %, ≤3 wt %, ≤1 wt %, or ≤0.5 wt % of the carbon dioxide present in the deethanizer overhead in line 412. In other examples, the treated deethanizer overhead in line 347 can be free of any carbon dioxide.

The sorbent can be or can include one or more adsorbent materials, absorbent materials, a mixture thereof, or a combination thereof. In some examples, the sorbent can be or can include at least one metal from Group 1, 10, or 11 of the periodic table of elements or an oxide thereof. Conventional process conditions can be used within the carbonyl sulfide removal unit 345, but the invention is not limited thereto.

It is to be understood that any given system 100, 200, 300, and 400, e.g., 300, described herein can include any one or more additional process units or stages described with reference to one or more of the other systems, e.g., 100, 200, and/or 400. It should also be understood that well-known process equipment or units have been left out for simplicity and ease of description. For example, the systems 100, 200, 300, and/or 400 can include a number of compressors, pumps, reboilers, heat exchangers, storage tanks, etc., which are readily apparent those skilled in the art.

In at least some examples, the first bottoms in line 123 in systems 200, 300 and 400 can be further processed as described to produce the overhead condensate in line 164 as described with reference to FIG. 1. It should be understood, however, that the aqueous mixture in line 147 may or may not be contacted with the neutralizing agent in line 150 in systems 200, 300, and/or 400 to neutralize acetic acid. CO₂, and H₂S that may or may not be present in the aqueous mixture in line 147. In some examples, a mass flow rate of the overhead condensate in line 164 that can be separated in systems 200, 300, and/or 400 can be about 1%, about 3%, about 5%, or about 7% to about 10%, about 12%, about 15%, about 17%, or about 20% of a mass flow rate the aqueous mixture in line 147 separated from the first bottoms in line 123. In other examples, the mass flow rate of the overhead condensate in line 164 that can be separated in systems 200, 300, and/or 400 can be about 1% to about 20%, about 3% to about 15%, about 5% to about 10% about 4% to about 12%, or about 3% to about 15% the mass flow rate the aqueous mixture in line 147 separated from the first bottoms in line 123.

This disclosure can further include at least the following non-limiting aspects and/or embodiments:

A1. A process for upgrading a hydrocarbon, comprising:
determining an amount of acetic acid that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof;
providing the hydrocarbon feed;
steam cracking the hydrocarbon feed to produce the steam cracker effluent;
separating a tar product, a steam cracker quench oil, and a first overhead from the steam cracker effluent, wherein the first overhead comprises pygas, acetic acid, and a process gas comprising ethylene;
separating a second overhead comprising the process gas and a first naphtha cut comprising the pygas, water, and acetic acid from the first overhead;
separating a pygas product and a first aqueous mixture comprising acetic acid from the first naphtha cut; and
contacting the first aqueous mixture with a predetermined amount of a neutralizing agent sufficient to produce a treated mixture comprising neutralized acetic acid.

A2. The process of A1, wherein the step of providing the hydrocarbon feed comprises:
providing a raw feed comprising naphthenic acid(s);
separating the raw feed in a flashing drum to produce an overhead vapor and a heavy bottoms liquid; and
supplying at least a portion of the overhear vapor as at least a portion of the hydrocarbon feed.

A3. The process of A1 or A2, wherein the raw feed has a total acid number (“TAN”) ≥0.5, or ≥1.0, or ≥1.5, or ≥2.0, or ≥2.5.

A4. The process of any of A2 or A3, wherein at least 5 wt % of the totality of the naphthenic acid(s) in the raw feed is distributed into the heavy bottoms liquid.

A5. The process of A4, wherein up to 20 wt % of the totality of the naphthenic acid(s) in the raw feed is distributed into the heavy bottoms liquid.

A6. The process of any of A1 to A5, further comprising:
heating the treated mixture to remove hydrogen sulfide, ammonia, or a mixture thereof to produce a third overhead and a second bottoms, wherein the third overhead comprises steam and at least one of hydrogen sulfide and ammonia, and wherein the second bottoms comprises an aqueous mixture comprising neutralized acetic acid;
heating the second bottoms to produce dilution steam;
separating the dilution steam and a third bottoms comprising neutralized acetic acid; and
removing the third bottoms from the process as a process water.

A7. The process of A6, further comprising:
condensing at least a portion of the third overhead to produce an overhead condensate; and
removing the overhead condensate from the process.

A8. The process of A7, wherein a mass flow rate of the overhead condensate is about 1% to about 100% of a mass flow rate the third overhead.

A9. The process of A7 or A8, wherein a mass flow rate of the overhead condensate is about 5% to about 10% of a mass flow rate the first aqueous mixture is separated from the first naphtha cut.

A10. The process of any of A6 to A9, wherein the third bottoms comprises ≥95 wt % of the acetic acid in steam cracker effluent in neutralized form.

A11. The process of any of A6 to A10, wherein the third bottoms comprises ≥99 wt % of the acetic acid in steam cracker effluent in neutralized form.

A12. The process of any of A1 to A11, wherein the neutralizing agent comprises one or more of: ammonia; a primary amine; a second amine; a tertiary amine; a diamine; and a caustic.

B1. A process for upgrading a hydrocarbon, comprising:
determining an amount of carbon monoxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof;
steam cracking the hydrocarbon feed to produce the steam cracker effluent;
separating a tar product, a steam cracker quench oil, and a first overhead from the steam cracker effluent, wherein the first overhead comprises pygas and a process gas comprising ethylene and carbon monoxide;
separating a second overhead comprising the process gas and a naphtha cut comprising the pygas from the first overhead;
separating a hydrogen-rich gas from the second overhead, wherein the hydrogen-rich gas comprises a first portion of the carbon monoxide contained in the steam cracker effluent; and feeding at least a portion of the hydrogen-rich gas into a methanator having a predetermined size and/or a pressure swing adsorption unit having a predetermined size, thereby collectively abating at least 90 wt % of the first portion of the carbon monoxide in the hydrogen-rich gas.

B2. The process of B1, wherein the hydrogen-rich gas comprises about 40 wt % to about 50 wt % of the carbon monoxide contained in the steam cracker effluent.

B3. The process of B1 or B2, further comprising separating a fuel gas from the process gas, wherein the fuel gas comprises molecular hydrogen, methane, and a second portion of the carbon monoxide contained in the steam cracker effluent.

B4. The process of B3, wherein the fuel gas comprises about 50 wt % to about 60 wt % of the carbon monoxide contained in the steam cracker effluent.

B5. The process of any of B1 to B4, wherein the hydrogen-rich gas is introduced into the methanator having a predetermined size, and wherein the methanator comprises a catalyst disposed therein, and wherein the catalyst comprises nickel, rhodium, ruthenium, or a mixture thereof.

B6. The process of any of B1 to B5, wherein the hydrogen-rich gas is introduced into the methanator, and wherein the predetermined size of the methanator is sufficient to convert ≥95 wt % of the first portion of carbon monoxide to methane.

B7. The process of any of B1 to B6, wherein at least a portion the carbon monoxide is produced by decomposing methanol contained in the hydrocarbon feed.

B8. The process of any of B1 to B7, wherein the hydrogen-rich gas is introduced the pressure swing adsorption unit having the predetermined size.

B9. The process of any of B1 to B8, wherein a purified hydrogen gas product is recovered from the methanator and/or the pressure swing adsorption unit, and wherein the purified hydrogen gas product comprises≤0.5 wt % of the carbon dioxide contained in the steam cracker effluent.

C1. A process for upgrading a hydrocarbon, comprising:
determining an amount of carbon dioxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof;
installing an amine unit having a predetermined capacity, the amine unit configured to remove a minimum amount of carbon dioxide from a second overhead comprising ethylene and carbon dioxide, wherein the predetermined capacity of the amine unit is based, at least in part, on the determined amount of carbon dioxide that will be present in the steam cracker effluent;
installing an amine regeneration unit having a predetermined capacity, the amine regeneration unit configured to regenerate a spent amine produced in the amine unit;
steam cracking the hydrocarbon feed to produce the steam cracker effluent;
separating a tar product, a steam cracker quench oil, and a first overhead from the steam cracker effluent, wherein the first overhead comprises pygas and a process gas;
separating a second overhead comprising the process gas and carbon dioxide and a first naphtha cut comprising the pygas from the first overhead;
contacting the second overhead with an amine within the amine unit having the predetermined size to remove at least 75 wt % of the carbon dioxide in the second overhead by producing a spent amine comprising a reaction product of the amine and the carbon dioxide;
separating a third overhead comprising ethylene and carbon dioxide and a second bottoms comprising the spent amine from the amine unit; and
introducing the second bottoms into the amine regeneration unit to produce a regenerated amine and carbon dioxide.

C2. The process of C1, further comprising:
installing a caustic unit having a predetermined capacity, the caustic unit configured to remove a minimum amount of carbon dioxide from the third overhead; and
contacting the third overhead within the caustic unit having the predetermined capacity to remove at least 75 wt % of the carbon dioxide in the third overhead by producing a spent caustic comprising a reaction product of the caustic and the carbon dioxide, wherein the predetermined capacity of the caustic unit is based, at least in part, on the determined amount of carbon dioxide that will be present in the steam cracker effluent; and separating a fourth overhead from the caustic unit, wherein the fourth overhead comprises ethylene and <7 wt % of the carbon dioxide contained in the steam cracker effluent.

C3. The process of C1 or C2, wherein ≥85 wt % of the carbon dioxide in the second overhead is removed in the amine unit, wherein ≥85 wt % of the carbon dioxide in the third overhead is removed in the caustic unit, and wherein the fourth overhead comprises <2.5 wt % of the carbon dioxide contained in the steam cracker effluent.

C4. The process of any of C1 to C3, wherein the amine comprises monoethanol amine, diethanol amine, or a mixture thereof.

D1. A process for upgrading a hydrocarbon, comprising:
determining an amount of carbon dioxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof;
preparing an aqueous amine solution having a predetermined concentration of amine, wherein the predetermined concentration of the amine in the aqueous amine solution is based, at least in part, on the determined amount of carbon dioxide that will be present in the steam cracker effluent;
steam cracking the hydrocarbon feed to produce a steam cracker effluent;
separating a tar product, a steam cracker quench oil, and a first overhead from the steam cracker effluent, wherein the first overhead comprises pygas and a process gas comprising ethylene and carbon dioxide;
separating a second overhead comprising the process gas and carbon dioxide and a first naphtha cut comprising the pygas from the first overhead;
contacting the second overhead with the aqueous amine solution having the predetermined concentration of amine to produce a treated mixture that can include an amine treated second overhead and a spent aqueous amine comprising an amine salt;
separating a third overhead comprising the amine treated second overhead and a second bottoms comprising the spent aqueous amine from the treated mixture, wherein at least 75 wt % of the carbon dioxide in the process gas is removed with the spent aqueous amine in the form of the amine salt.

D2. The process of D1, further comprising:
preparing an aqueous caustic solution having a predetermined concentration of caustic, wherein the predetermined concentration of the caustic in the aqueous caustic solution is based, at least in part, on the determined amount of carbon dioxide that will be present in the steam cracker effluent; and
contacting the third overhead with the aqueous caustic solution to produce a caustic treated third overhead and a spent caustic solution comprising a carbonate salt; and
separating a fourth overhead comprising the caustic treated third overhead and a third bottoms comprising the spent caustic solution, wherein at least 75 wt % of the carbon dioxide in the third overhead is removed with the spent caustic in the form of the carbonate salt.

D3. The process of D1 or D2, further comprising regenerating the spent aqueous amine in the third bottoms to produce a regenerated amine and carbon dioxide.

D4. The process of any of D1 to D3, wherein ≥85 wt % of the carbon dioxide in the third overhead is removed.

D5. The process of any of D1 to D4, wherein the amine comprises monoethanol amine, diethanol amine, or a mixture thereof, and wherein the caustic comprises sodium hydroxide, potassium hydroxide, or a mixture thereof.

E1. A process for upgrading a hydrocarbon, comprising:
determining an amount of carbon dioxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof,
introducing a sufficient amount of a sorbent into a carbonyl sulfide removal unit to allow the carbonyl sulfide removal unit to process a depropanizer overhead that is to be separated from the steam cracker effluent for at least as long as a predetermined period of time without requiring replacement or re-activation of the sorbent due to deactivation caused by carbon dioxide present in the depropanizer overhead.

E2. The process of E1, further comprising:
steam cracking the hydrocarbon feed to produce a steam cracker effluent;
separating an overhead comprising carbon dioxide, methanol, ethane, ethylene, propane, and propylene from the steam cracker effluent;
separating the depropanizer overhead comprising carbon dioxide from the process gas; and
introducing the depropanizer overhead into the carbonyl sulfide removal unit for at least the predetermined period of time.

E3. The process of E1 or E2, wherein the predetermined period of time is at least 5 days.

E4. The process of any of E1 to E3, wherein the sorbent is an adsorbent.

E5. The process of any of E1 to E4, wherein the sorbent comprises at least one metal from Group 1, 10, or 11 of the periodic table of elements or an oxide thereof.

F1. A process for upgrading a hydrocarbon, comprising:
determining an amount of carbon dioxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof,
introducing a sufficient amount of a sorbent into a carbonyl sulfide removal unit to allow the carbonyl sulfide removal unit to process a deethanizer overhead that is to be separated from the steam cracker effluent for at least as long as a predetermined period of time without requiring replacement or re-activation of the sorbent due to deactivation caused by carbon dioxide present in the deethanizer overhead.

F2. The process of F1, further comprising:
steam cracking the hydrocarbon feed to produce a steam cracker effluent;
separating an overhead comprising carbon dioxide methanol, ethane, ethylene, propane, and propylene from the steam cracker effluent;
separating the deethanizer overhead comprising carbon dioxide from the process gas; and
introducing the deethanizer overhead into the carbonyl sulfide removal unit for at least the predetermined period of time.

F3. The process of F1 or F2, wherein the predetermined period of time is at least 5 days.

F4. The process of any of F1 to F3, wherein the sorbent comprises at least one metal from Group 1, 10, or 11 of the periodic table of elements or an oxide thereof.

F5. The process of any of F1 to F4, wherein the hydrocarbon feed has a total acid number of ≥0.5 mg KOH/g of hydrocarbon feed, as measured according to ASTM D664-18e2.

F6. The process of any of F1 to F5, wherein the hydrocarbon feed has a total acid number of ≥1.5 mg KOH/g of hydrocarbon feed, as measured according to ASTM D664-18e2.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

1. A process for upgrading a hydrocarbon, comprising:
determining an amount of acetic acid that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof;
providing the hydrocarbon feed;
steam cracking the hydrocarbon feed to produce the steam cracker effluent;
separating a tar product, a steam cracker quench oil, and a first overhead from the steam cracker effluent, wherein the first overhead comprises pygas, acetic acid, and a process gas comprising ethylene;
separating a second overhead comprising the process gas and a first naphtha cut comprising the pygas, water, and acetic acid from the first overhead;
separating a pygas product and a first aqueous mixture comprising acetic acid from the first naphtha cut; and
contacting the first aqueous mixture with a predetermined amount of a neutralizing agent sufficient to produce a treated mixture comprising neutralized acetic acid.

2. The process of claim 1, wherein the step of providing the hydrocarbon feed comprises:
providing a raw feed comprising naphthenic acid(s);
separating the raw feed in a flashing drum to produce an overhead vapor and a heavy bottoms liquid; and
supplying at least a portion of the overhead vapor as at least a portion of the hydrocarbon feed.

3. The process of claim 1, wherein the raw feed has a total acid number (“TAN”) ≥0.5.

4. The process of claim 2, wherein at least 5 wt % of the totality of the naphthenic acid(s) in the raw feed is distributed into the heavy bottoms liquid.

5. The process of claim 4, wherein up to 20 wt % of the totality of the naphthenic acid(s) in the raw feed is distributed into the heavy bottoms liquid.

6. The process of claim 1, further comprising:
heating the treated mixture to remove hydrogen sulfide, ammonia, or a mixture thereof to produce a third overhead and a second bottoms, wherein the third overhead comprises steam and at least one of hydrogen sulfide and ammonia, and wherein the second bottoms comprises an aqueous mixture comprising neutralized acetic acid;
heating the second bottoms to produce dilution steam;
separating the dilution steam and a third bottoms comprising neutralized acetic acid; and
removing the third bottoms from the process as a process water.

7. The process of claim 6, wherein the third bottoms comprises ≥95 wt % of the acetic acid in steam cracker effluent in neutralized form.

8. The process of claim 6, wherein the third bottoms comprises ≥99 wt % of the acetic acid in steam cracker effluent in neutralized form.

9. The process of claim 1, wherein the neutralizing agent comprises one or more of: ammonia; a primary amine; a secondary amine; a tertiary amine; a di-amine; and a caustic.

10. A process for upgrading a hydrocarbon, comprising:
determining an amount of carbon monoxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof;
steam cracking the hydrocarbon feed to produce the steam cracker effluent;
separating a tar product, a steam cracker quench oil, and a first overhead from the steam cracker effluent, wherein the first overhead comprises pygas and a process gas comprising ethylene and carbon monoxide;
separating a second overhead comprising the process gas and a bottoms comprising the pygas from the first overhead;
separating a hydrogen-rich gas from the second overhead, wherein the hydrogen-rich gas comprises a first portion of the carbon monoxide contained in the steam cracker effluent; and
feeding at least a portion of the hydrogen-rich gas into a methanator having a predetermined size and/or a pressure swing adsorption unit having a predetermined size, thereby collectively abating at least 90 wt % of the first portion of the carbon monoxide in the hydrogen-rich gas.

11. The process of claim 9, wherein the hydrogen-rich gas comprises about 40 wt % to about 50 wt % of the carbon monoxide contained in the steam cracker effluent.

12. The process of claim 10, further comprising separating a fuel gas from the process gas, wherein the fuel gas comprises molecular hydrogen, methane, and a second portion of the carbon monoxide contained in the steam cracker effluent.

13. The process of claim 12, wherein the fuel gas comprises about 50 wt % to about 60 wt % of the carbon monoxide contained in the steam cracker effluent.

14. The process of claim 10, wherein the hydrogen-rich gas is introduced into the methanator having a predetermined size, and wherein the methanator comprises a catalyst disposed therein, and wherein the catalyst comprises nickel, rhodium, ruthenium, or a mixture thereof.

15. The process of claim 10, wherein the hydrogen-rich gas is introduced into the methanator, and wherein at least 95 wt % of the first portion of the carbon monoxide is converted into methane in the methanator.

16. The process of claim 10, wherein at least a portion the carbon monoxide is produced by decomposing methanol contained in the hydrocarbon feed.

17. The process of claim 10, wherein the hydrogen-rich gas is introduced the pressure swing adsorption unit having the predetermined size.

18. The process of claim 17, wherein the pressure swing adsorption unit has a capacity to abate at least 90 wt % of the first portion of the carbon monoxide in the hydrogen-rich gas.

19. The process of claim 10, wherein a purified hydrogen gas product is recovered from the methanator, the pressure swing adsorption unit, or the combination thereof, and wherein the purified hydrogen gas product comprises ≤0.5 wt % of the carbon dioxide contained in the steam cracker effluent.

20. A process for upgrading a hydrocarbon, comprising:
determining an amount of carbon dioxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof;
installing an amine unit having a predetermined capacity, the amine unit configured to remove a minimum amount of carbon dioxide from a second overhead comprising ethylene and carbon dioxide, wherein the predetermined capacity of the amine unit is based, at least in part, on the determined amount of carbon dioxide that will be present in the steam cracker effluent;
installing an amine regeneration unit having a predetermined capacity, the amine regeneration unit configured to regenerate a spent amine produced in the amine unit;
steam cracking the hydrocarbon feed to produce the steam cracker effluent;
separating a tar product, a steam cracker quench oil, and a first overhead from the steam cracker effluent, wherein the first overhead comprises pygas and a process gas;
separating a second overhead comprising the process gas and carbon dioxide and a first naphtha cut comprising the pygas from the first overhead;
contacting the second overhead with an amine within the amine unit having the predetermined size to remove at least 75 wt % of the carbon dioxide in the second overhead by producing a spent amine comprising a reaction product of the amine and the carbon dioxide;
separating a third overhead comprising ethylene and carbon dioxide and a second bottoms comprising the spent amine from the amine unit; and
introducing the second bottoms into the amine regeneration unit to produce a regenerated amine and carbon dioxide.

21. The process of claim 20, further comprising:
installing a caustic unit having a predetermined capacity, the caustic unit configured to remove a minimum amount of carbon dioxide from the third overhead; and
contacting the third overhead within the caustic unit having the predetermined capacity to remove at least 75 wt % of the carbon dioxide in the third overhead by producing a spent caustic comprising a reaction product of the caustic and the carbon dioxide, wherein the predetermined capacity of the caustic unit is based, at least in part, on the determined amount of carbon dioxide that will be present in the steam cracker effluent; and
separating a fourth overhead from the caustic unit, wherein the fourth overhead comprises ethylene and <7 wt % of the carbon dioxide contained in the steam cracker effluent.

22. The process of claim 20, wherein ≥85 wt % of the carbon dioxide in the second overhead is removed in the amine unit, wherein ≥85 wt % of the carbon dioxide in the third overhead is removed in the caustic unit, and wherein the fourth overhead comprises <2.5 wt % of the carbon dioxide contained in the steam cracker effluent.

23. The process of claim 20, wherein the amine comprises monoethanol amine, diethanol amine, or a mixture thereof.

24. A process for upgrading a hydrocarbon, comprising:
determining an amount of carbon dioxide that will be present in a steam cracker effluent based, at least in part, on a composition of a hydrocarbon feed to be steam cracked, a temperature the hydrocarbon feed will be heated at during steam cracking, a residence time the hydrocarbon feed will be heated at the temperature during steam cracking, or a combination thereof;
preparing an aqueous amine solution having a predetermined concentration of amine, wherein the predetermined concentration of the amine in the aqueous amine solution is based, at least in part, on the determined amount of carbon dioxide that will be present in the steam cracker effluent;
steam cracking the hydrocarbon feed to produce a steam cracker effluent;
separating a tar product, a steam cracker quench oil, and a first overhead from the steam cracker effluent, wherein the first overhead comprises pygas and a process gas comprising ethylene and carbon dioxide;
separating a second overhead comprising the process gas and carbon dioxide and a first naphtha cut comprising the pygas from the first overhead;
contacting the second overhead with the aqueous amine solution having the predetermined concentration of amine to produce a treated mixture that can include an amine treated second overhead and a spent aqueous amine comprising an amine salt;
separating a third overhead comprising the amine treated second overhead and a second bottoms comprising the spent aqueous amine from the treated mixture, wherein at least 75 wt % of the carbon dioxide in the process gas is removed with the spent aqueous amine in the form of the amine salt.
